(19) United States
(12) Patent Application Publication
(10) Pub. No.: US 2023/0089654 A1
(43) Pub. Date: Mar. 23, 2023

(54) NK CELLS OR T CELLS EXPRESSING HEMATOPOIETIC GROWTH FACTOR RECEPTORS AND METHODS OF USE

(71) Applicant: The United States of America, as represented by the Secretary, Dept. of Health and Human Services, Bethesda, MD (US)

(72) Inventors: Richard W. Childs, Rockville, MD (US); David S.J. Allan, Rockville, MD (US); Chantiya Chanswangphuwana, Bangkok (TH)

(73) Assignee: The United States of America, as represented by the Secretary, Dept. of Health and Human Services, Bethesda, MD (US)

(21) Appl. No.: 17/821,703

(22) Filed: Aug. 23, 2022

Related U.S. Application Data
(63) Continuation of application No. PCT/US2021/019288, filed on Feb. 23, 2021.
(60) Provisional application No. 62/980,854, filed on Feb. 24, 2020.

(51) Int. Cl. A61K 35/17; A61P 35/00

(52) U.S. Cl. CPC A61K 35/17; A61P 35/00

(57) ABSTRACT

Modified natural killer (NK) or T cells expressing hematopoietic growth factor receptors are provided. In some embodiments, the NK cells or T cells express a thrombopoietin receptor or an erythropoietin receptor. Methods of treating a subject with cancer are also provided, including administering the modified NK cells or T cells to the subject in combination with a thrombopoietin receptor agonist or erythropoietin receptor agonist, and in some example, interleukin-2, particularly reduced or low-dose amounts of IL-2.

### CROSS REFERENCE TO RELATED APPLICATIONS

This application is a continuation of International Application No. PCT/US2021/019288 filed Feb. 23, 2021, which claims the benefit of U.S. Provisional Application No. 62/980,854, filed Feb. 24, 2020, both of which are incorporated herein by reference in their entirety.

### FIELD

This disclosure relates to modified natural killer or T cells expressing hematopoietic growth factor receptors and methods of their use.

### SEQUENCE LISTING INCORPORATION BY REFERENCE

The Sequence Listing is submitted as an XML file in the form of the file named 4239-104153-03_Sequence_Listing.xml, which was created on Aug. 21, 2022, and is 29,351 bytes, which is incorporated by reference herein.

### BACKGROUND

There currently exist a number of different pre-clinical and clinical strategies for utilizing natural killer (NK) cells as an immunotherapeutic to treat cancer. NK cells lack the requirement for prior-sensitization and can induce tumor cytotoxicity in an antigen-independent manner without causing graft-versus-host disease, making NK cells an attractive cell-based treatment option. Recent advances in genetic engineering techniques now provide the possibility to modify NK cells to enhance their efficacy in treating cancer. In this regard, several pre-clinical reports have shown that the antitumor cytotoxicity of NK cells can be improved by engineering them to target specific tumor antigens. One significant limitation of NK cell based immunotherapy is their reliance on cytokines, such as interleukin (IL)-2, IL-15, or IL-21, which enhance NK cell persistence and expansion in vitro and in vivo. At present, most clinical trials evaluating NK cell therapy in humans utilize the exogenous administration of IL-2 or IL-15 following adoptive cell transfer (Childs and Carlsten, Nat. Rev. Drug Discov. 14:487-498, 2015). Although IL-2 promotes activation, proliferation and cytotoxicity of NK cells, it also has the undesirable effect of inducing the expansion of regulatory T cells (Treg) which may suppress immune responses. Although IL-15 improves NK cell homeostasis, expansion and cytolytic capacity without expanding Tregs (Hitchcock and Kaushansky, Br. J. Maematol. 165:259-268, 2014; Rautela and Huntington, Curr. Opin. Immunol. 44:1-6, 2017; Macais et al., Nat. Immunol. 15:749-757, 2014), it has cytokine-associated toxicities similar to those observed with IL-2, including capillary leak syndrome, hypotension, fever, and chills.

Investigators have recently explored a number of approaches to improve NK cell persistence in vivo that minimize toxicities and reduce or avoid the need for exogenous cytokine administration. Genetic modification of NK cell lines to express IL-2 or endoplasmic reticulum-retained IL-2 have been shown to result in autonomous NK cell proliferation (Nagashima et al, Blood 91:3850-3861, 1998; Konstantinidis et al., Exp. Hematol. 33:159-164, 2005). Likewise, expressing IL-15 or membrane-bound IL-15 on NK cells can enhance their proliferation and cytotoxicity in absence of exogenous cytokines (Zhang et al., Haematologica 89:338-347, 2004; Sahm et al., Cancer Immunol. Immunother. 61:1451-1461, 2012; Imamura et al., Blood 124:1081-1088, 2014). Recently, Liu et al demonstrated that NK cells derived from cord blood engineered to express anti-CD19 CAR, IL-15, and a suicide gene had improved anti-tumor activity and long-term persistence (Leukemia 32:520-531, 2018). In addition, an IL-15 superagonist complex called ALT-803 exhibited a longer half-life and better immune NK cell activation in vivo compared with wild-type IL-15 (Xu et al., Cancer Res. 73:3075-3086, 2013; Romee et al., Blood 131:2515-2527, 2018).

### SUMMARY

There remains a need to improve NK cell survival, proliferation, and cytotoxicity, while decreasing undesirable or adverse effects. Disclosed herein are modified immune cells (such as NK cells or T cells) expressing hematopoietic growth factor receptors, and methods of their use.

In some embodiments, modified NK cells or T cells expressing a heterologous thrombopoietin receptor (c-MPL) protein or a heterologous erythropoietin receptor (EPOR) protein are provided. In particular examples, the c-MPL or EPOR protein is a human protein. Also provided are modified NK cells or T cells including a nucleic acid encoding the c-MPL protein or EPOR protein. The modified NK cell or T cell may be a human NK cell or T cell. In additional embodiments, the modified NK cell or T cell further expresses a chimeric antigen receptor (CAR).

Methods of culturing or expanding the modified NK cells or T cells are also provided. In some examples, modified NK cells or T cells expressing c-MPL are cultured in the presence of a thrombopoietin receptor agonist. In other examples, modified NK cells or T cells expressing EPOR are cultured in the presence of an erythropoietin receptor agonist. In some embodiments, the modified NK cells or T cells are cultured in the presence of a thrombopoietin receptor agonist or erythropoietin receptor agonist and interleukin (IL)-2. In particular examples the amount of IL-2 is a reduced amount of IL-2 (“low dose”), which in some examples, is about 1-50 U/ml IL-2 (for example, about 1-5 U/ml, about 2-10 U/ml, about 5-15 U/ml, about 10-25 U/ml, about 20-40 U/ml, about 25-50 U/ml, or about 5-25 U/ml).

Also provided are methods of treating a subject with cancer. In some examples, the methods include administering modified NK cells or T cells expressing a heterologous thrombopoietin receptor (c-MPL) protein to the subject and administering a c-MPL agonist to the subject (such as TPO, eltrombopag, avatrombopag, or romiplostim). In some examples, the method also includes administering IL-2 to the subject, such as “low dose” IL-2 (e.g., about 0.5-4 million units/m²). In some examples, the methods also include contacting the modified NK cells or T cells with IL-2 (e.g., about 1-50 U/ml IL-2 (for example, about 1-5 U/ml, about 2-10 U/ml, about 5-15 U/ml, about 10-25 U/ml, about 20-40 U/ml, about 25-50 U/ml, or about 5-25 U/ml) prior to administering the NK cells or T cells to the subject.

In further examples, the methods include administering modified NK cells or T cells expressing a heterologous EPOR protein to the subject and administering an EPOR agonist to the subject (such as erythropoietin or darbepoetin). In some examples, the method also includes administering IL-2 to the subject, such as “low dose” IL-2 (e.g., about 0.5-4 million units/m²). In some examples, the methods also include contacting the modified NK cells or T cells with IL-2 (e.g., about 1-50 U/ml IL-2 (for example, about 1-5 U/ml, about 2-10 U/ml, about 5-15 U/ml, about 10-25 U/ml, about 20-40 U/ml, about 25-50 U/ml, or about 5-25 U/ml) prior to administering the NK cells or T cells to the subject.

The foregoing and other features of the disclosure will become more apparent from the following detailed description, which proceeds with reference to the accompanying figures.

### BRIEF DESCRIPTION OF THE DRAWINGS

FIGS. 1A-1H show exogenous expression of EPOR or c-MPL in NK cell lines facilitates their proliferation in the presence of EPO or TPO. EPOR and GFP (FIG. 1A) or c-MPL and GFP (FIG. 1E) expression on NK-92 cell lines after fluorescence-activated cell sorting of transduced cells. Fold expansion of EPOR⁺ NK-92 cells (FIGS. 1B-1D) or c-MPL⁺ NK-92 cells (FIGS. 1F-1H) in the presence of IL-2, EPO, or TPO supplemented into culture medium at varying concentrations as indicated. Mean and standard deviation (SD) from 3-4 independent experiments are shown. Significance was analyzed at day 8 using unpaired Student's t-test. (*p<0.05, **p<0.01, ****p<0.0001).

FIGS. 2A-2B illustrate that EPO and TPO do not affect the unmodified NK-92 cell line. Histograms show EPOR and c-MPL expression on parental NK-92 cells (FIG. 2A). The fold expansion of parental NK-92 cells when cultured with the indicated concentrations of IL-2/EPO/TPO are shown (FIG. 2B). Data represent 3 independent experiments.

FIGS. 3A-3H show exogenous expression of EPOR or c-MPL in KHYG-1 NK cell line facilitates proliferation in the presence of EPO or TPO. EPOR and GFP (FIG. 3A) or c-MPL and GFP (FIG. 3E) expression on KHYG-1 cell lines after fluorescence-activated cell sorting of transduced cells. Fold expansion of EPOR⁺ KHYG-1 cells (FIGS. 3B-3D) or c-MPL⁺ KHYG-1 cells (FIGS. 3F-3H) in the presence of IL-2, EPO, or TPO supplemented into culture medium at varying concentrations as indicated. Mean and standard deviation (SD) from 3-4 independent experiments are shown. Significance was analyzed at day 8 using unpaired Student's t-test. (*p<0.05).

FIGS. 4A-4B demonstrate that EPO and TPO do not affect the unmodified KHYG-1 cell line. Histograms show EPOR and c-MPL expression on parental KHYG-1 cells (FIG. 4A). The fold expansion of parental KHYG-1 cells when cultured with the indicated concentrations of IL-2/EPO/TPO are shown (FIG. 4B). Data represent 3 independent experiments.

FIGS. 5A-5D illustrate that EPOR and c-MPL expressing NK-92 cell lines show lower levels of apoptosis and cell death when stimulated with their ligands. EPOR⁺ NK-92 cells (FIGS. 5A and 5B) and c-MPL⁺ NK-92 cells (FIGS. 5C and 5D) were cultured in different conditions as indicated. FIGS. 5A and 5C show fold expansion of viable cells. FIGS. 5B and 5D show the percentage of 7-AAD⁺ and/or AnnexinV⁺ cells detected by flow cytometry at day 2 or 3 of culture. **p<0.01, ***p<0.001 and ****p<0.0001, by unpaired Student's t-test from 3 independent experimental results.

FIGS. 6A-6B show that phosphorylated p44/p42 MAPK did not increase in cytokine treated NK-92 cell lines. Flow cytometry measurements of p-p44/42 MAPK expression in EPOR⁺ NK-92 cells (FIG. 6A) and c-MPL⁺ NK-92 cells (FIG. 6B) were presented as RMFI (normalized to isotype control) separately for each type of activation shown on the X-axis. (PMA; phorbol 12-myristate 13-acetate).

FIGS. 7A-7H show signaling pathways in EPOR and c-MPL expressing NK-92 cells that increase levels of anti-apoptotic proteins. EPOR⁺ NK-92 cells (FIGS. 7A-7D) and c-MPL⁺ NK-92 cells (FIGS. 7E-7H) were cultured without IL-2 overnight, then incubated with different IL-2, EPO, TPO concentrations for 15 minutes before analysis of pSTAT5 and pS6 or for 24 hours before Bcl-2 and Bcl-xL analysis by flow cytometry. The relative mean fluorescent intensity (RMFI) of the protein expression in EPOR⁺ NK-92 (FIGS. 7A-7D) and c-MPL⁺ NK-92 (FIGS. 7E-7H) cells are displayed with SD according to the experimental conditions indicated. RMFI was normalized to isotype control in each case. *p<0.05, **p<0.01, ***p<0.001 and ***p<0.0001, by unpaired Student's t-test from 3 independent experiments.

FIGS. 8A-8C show evaluation of signaling pathways and anti-apoptotic proteins in EPO-treated EPOR+ NK-92 cells. This figure shows further data from the experiment depicted in FIGS. 5A-5D. On day 2, cells were analyzed for (FIG. 8A) Bcl-2 and (FIG. 8B) Bcl-xL expression (RMFI) by flow cytometry. In parallel, the pSTAT5 and pS6 expression were determined in EPOR⁺ NK-92 cells from day 2 and 3 of the experiment (FIG. 8C).

FIGS. 9A-9C show evaluation of signaling pathways and anti-apoptotic proteins in TPO-treated c-MPL⁺ NK-92 cells. This figure shows further data from the same experiment depicted in FIGS. 5A-5D. On day 2, the cells were analyzed for (FIG. 9A) Bcl-2 and (FIG. 9B) Bcl-xL expression (RMFI). In parallel, the pSTAT5 and pS6 expression were determined in c-MPL⁺ NK-92 cells from day 2 and 3 of the experiment (FIG. 9C).

FIGS. 10A-10B demonstrate that c-MPL activated NK-92 cells show significantly greater cytotoxic activity in vitro vs Raji tumor cells. EPOR⁺ NK-92 and c-MPL⁺ NK-92 were cultured in different media conditions as indicated for 24-40 hours before performing cytotoxicity assays. The mean (and SD) percentages of non-viable Raji, K562, or 721.221 target cells after 4-hour incubation with (FIG. 10A) EPOR⁺ NK-92 or (FIG. 10B) c-MPL⁺ NK-92 effector cells are presented for each effector-to-target ratio (E:T). (*p<0.05 by unpaired Student's t-test of 4-5 independent experiments).

FIGS. 11A-11F show effects on proliferation of primary human NK cells genetically modified to express EPOR or c-MPL. Human NK cells were stimulated with irradiated SMI-LCL feeder cells and IL-2 for 4-5 days then underwent lentiviral transduction. Three days later, transduced cells were examined for relative transgene expression by flow cytometry (FIG. 11A) (data from one donor) and were cultured with varying concentrations of the indicated cytokines (FIGS. 11B-11F). The activated but untransduced (Mock) NK cells expanded in an IL-2 dependent manner (FIG. 11B). The fold-expansion of GFP⁺ (virally-transduced) NK cells expressing EPOR (FIGS. 11C-11D) and c-MPL (FIGS. 11E-11F) is shown. Statistical significance with samples cultured in varying media was measured with unpaired student's t-test using data from 3 healthy donors on day 6 of culture (equivalent to day 14 from the initial NK cell isolation (D+14). (*<0.05).

FIGS. 12A-12B show that the expansion of transduced primary NK cells was influenced by cytokines and increased in presence of TPO. Further data from the same experiment depicted in FIGS. 11A-11F. The percentage of (FIG. 12A) EPOR-transduced or (FIG. 12B) c-MPL-transduced NK cells indicated by GFP expression are shown relative to cytokines and culture duration.

FIGS. 13A-13D demonstrate that transduced c-MPL receptors augment the function of primary human NK cells. Human NK cells were stimulated and transduced with c-MPL/GFP lentiviral vectors. 2-3 days later, cells were subdivided into cultures containing IL-2 and/or TPO at the indicated concentrations. After 4 days, cellular function was assessed by mixing at a 1:1 ratio with K562 target cells and assessing (FIG. 13A) NK cell degranulation (by surface anti-CD107a staining) two hours later, or (FIG. 13B) intracellular IFN-γ and TNF-α four hours after addition of K562. Results for gated GFP⁺ transduced cells or independent untransduced cultures from the same donors are shown. The cell surface phenotype of the NK cells was also assessed after 4 days of culture with cytokines as indicated (FIG. 13C). To control for inter-donor variation, percentages of lymph node homing receptor CD62L⁺ cells were normalized to values obtained in untransduced NK cells cultured with 500 U/ml IL-2 from each donor (FIG. 13D). *p<0.05 by paired Student's t-test of 4 independent experiments.

FIGS. 14A-14B show that c-MPL-expressing human NK cells persist preferentially in immunodeficient mice administered TPO. NK cells from human PBMC were stimulated for 4 days with LCL feeder cells and IL-2, then transduced with matched lentiviral vectors encoding GFP-c-MPL or GFP-CD34 as a control. Cells were maintained in IL-2-containing medium for 3-4 additional days and averaged approximately 20% GFP⁺. Both populations were then mixed at a 1:1 ratio immediately before injection intravenously into NSG mice (such that GFP MPL⁺ cells and GFP⁺ CD34⁺ cells each constituted ˜10% of the mixed cells). Each mouse received 2.2×10⁷ total human NK cells. Mice received daily injections of human TPO (50 μg/kg) or human IL-2 (10,000 IU/mouse) for 4 days, after which, presence of transduced human cells was quantified in various organs via flow cytometry. MPL-transduced NK cells were identified as GFP⁺ MPL⁺ while CD34-transduced NK cells were gated as GFP CD34⁺. The frequency of transduced cells is presented (FIG. 14A) as a percentage of MHC-I⁺ human cells isolated from organs or (FIG. 14B) as percent of total live mononuclear cells recovered from each organ. The legend applies to both FIGS. 14A and 14B. Data from 9 mice per group are shown with NK cells derived from 3 human blood donors. Data was normalized to account for unequal transduction or subset mixing before injection using the equation:

$$\frac{Percentage of {cells}_{organ}}{Percentage of {cells}_{infused cell mix}} \times Mean ( Percentage of {cells}_{infused cell mix} )$$

(the last term representing the mean of all MPL⁺ and CD34⁺ groups from all donors). Groups were compared by paired T-test, as indicated. The significance of indicated comparisons was unaffected by data normalization (with the exception of bone marrow samples).

FIGS. 15A-15D show effects on proliferation of primary human T cells genetically modified to express EPOR or c-MPL. PBMC were transduced after 2 days of stimulation with anti-CD3 and anti-CD28 antibodies. After 4 additional days, CD3⁺ T-cells were examined for the transduced gene expression by flow cytometry (FIG. 15A) (data from one donor) and cultured with varying concentrations of the indicated cytokines (FIGS. 15B-15D). The activated but untransduced (Mock) T cells expanded in an IL-2 dependent manner (FIG. 15B). The fold-expansion of GFP⁺ (virally-transduced) T cells expressing (FIG. 15C) EPOR and (FIG. 15D) c-MPL is shown. Statistical significance with samples cultured in varying media as measured with unpaired student's t-test using data from 4 healthy donors on day 6 of culture (equivalent to day 12 from the initial T-cell isolation (D+12)). (**<0.01).

FIGS. 16A-16C show proliferation of human NK cells transduced to express GFP and c-MPL cultured for 4-5 days with the indicated concentrations of thrombopoietin (FIG. 16A), romiplostim (FIG. 16B), or eltrombopag (FIG. 16C). Numbers of GFP⁺ cells were enumerated and expressed as a percentage of numbers obtained from parallel cultures containing 500 U/mL IL-2. Mean and SEM of results from 3 blood donors are shown.

### SEQUENCE LISTING

Any nucleic acid and amino acid sequences listed herein or in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is the amino acid sequence of an exemplary thrombopoietin receptor (c-MPL).

SEQ ID NO: 2 is an exemplary nucleic acid sequence encoding the thrombopoietin receptor.

SEQ ID NO: 3 is the amino acid sequence of an exemplary erythropoietin receptor.

SEQ ID NO: 4 is an exemplary nucleic acid sequence encoding the erythropoietin receptor.

SEQ ID NO: 5 is the nucleic acid sequence of an exemplary vector including a nucleic acid encoding a thrombopoietin receptor.

SEQ ID NO: 6 is the nucleic acid sequence of an exemplary vector including a nucleic acid encoding an erythropoietin receptor.

### DETAILED DESCRIPTION

Hematopoietic growth factors, such as recombinant human erythropoietin (EPO) and thrombopoietin (TPO) mimetics are FDA-approved for use in humans with anemia and thrombocytopenia and have been associated with minimal adverse events. Binding of EPO to its erythropoietin receptor (EPOR) promotes differentiation, proliferation and survival of erythroid progenitor cells. TPO, a ligand of the c-MPL receptor, is important for megakaryocyte differentiation and expansion as well as hematopoietic stem cell proliferation and maintenance. Both the EPO/EPOR and TPO/c-MPL interactions have been shown to transduce signals through three-main pathways; JAK-STAT, PI3K-AKT and MAPK pathways, having similarities to IL-2 and IL-15 signaling cascades.

The data presented herein demonstrate that genetic manipulation of NK cells or T cells to exogenously express hematopoietic growth factor receptors can provide adjuvant signals to improve their proliferative capacity, tumor cytotoxicity, and survival while simultaneously reducing their requirement for IL-2. In the presence of relatively low doses of IL-2, the cell yields of both EPOR and c-MPL expressing NK-92 NK cells were improved when cells were cultured in the presence of EPO or TPO respectively. Similar effects were also observed in the KHYG-1 cell line, albeit these effects were less pronounced, perhaps due to its higher baseline proliferative rate and greater dependence on higher concentrations of IL-2. In particular examples, enhanced tumor cytotoxicity and an augmentation in proliferation in primary human NK cells transduced to express the c-MPL receptor was observed.

In the data presented herein, TPO concentrations analogous to those obtained pharmacologically in humans were utilized. Remarkably, proliferation was augmented in c-MPL⁺ transduced NK cells cultured in TPO plus 25 U/ml of IL-2 to a level comparable to that observed in NK cell populations cultured in 20-fold higher doses of IL-2 (500 U/ml). Although EPO administration enhanced the proliferative capacity of EPOR transduced NK-92 cells, it did not augment the proliferation of EPOR transduced primary NK cells, possibly due to limitations in the experiments shown herein, where surface expression of the EPOR on primary NK cells appeared to not be induced to the higher levels achieved with the c-MPL receptor. In receptor transduced NK92 cells, TPO or EPO ligands clearly showed additive or synergistic effects on proliferation, survival, and cytotoxicity when combined with IL-2.

Stimulating c-MPL and IL-2 receptor together on transduced NK-92 cells augmented the anti-tumor function against Raji cells, which remarkably was superior to IL-2 alone, even when cells were cultured in high doses of this cytokine (200 U/ml). Primary NK cells efficiently recognize K562 targets, and MPL-transduced human NK cells co-incubated with K562 showed striking functional increases in degranulation and cytokine production in response to TPO administration. These data suggest that MPL ligation in NK cells with transgenic c-MPL expression could facilitate NK cell activation and tumor killing.

An unexpected finding was that primary NK cells engineered to express c-MPL and treated with TPO showed higher percentages of the lymphoid tissue homing receptor CD62L (L-selectin). IL-2 activation and ex vivo expansion have previously been shown to down-regulate CD62L.

The results presented herein in immunodeficient mice indicate that c-MPL-transduced primary NK cells have superior in vivo survival in response to systemic TPO administration compared with control populations. The numbers of MPL NK cells recovered in TPO-treated mice approached those detected in mice administered IL-2. These data may be translated to a clinical trial setting, including infusion of genetically modified c-MPL expressing NK cells followed by administration of an FDA approved TPO-mimetic (e.g. Eltrombopag, Romiplostim) and combined with low dose IL-2. Such regimens might avoid the substantial side effects caused by intermediate or high-dose IL-2, while inducing a selective cellular proliferative effect that is restricted to the genetically modified NK cells, avoiding the induction of proliferation of regulatory T-cells which inhibit the antitumor effects of NK and T-cells.

### I. Terms

Unless otherwise noted, technical terms are used according to conventional usage. Definitions of common terms in molecular biology may be found in Lewin's Genes X, ed. Krebs et al., Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, Proteomics and Informatics, 3^{rd} Edition, Springer, 2008 (ISBN: 1402067534), and other similar references.

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. All publications, patent applications, patents, and other references mentioned herein are incorporated by reference in their entirety, as are the GenBank Accession numbers, as present on the filing date of this application. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Autologous: Refers to tissues, cells or nucleic acids taken from an individual's own tissues. For example, in an autologous transfer or transplantation of NK cells or T cells, the donor and recipient are the same person. Autologous (or “autogeneic” or “autogenous”) is related to self, or originating within an organism itself.

Erythropoietin (EPO): Erythropoietin is a hematopoietic growth factor that stimulates red blood cell production in the bone marrow. It is produced by the kidneys and secreted into the blood. EPO expression is upregulated by hypoxia, leading to increased red blood cell production and oxygen carrying capacity in the blood. Exemplary human EPO sequences include GenBank Accession No. NM_000799 (nucleic acid) and NP_000790 (amino acid). However, one of ordinary skill in the art can identify additional EPO sequences.

Erythropoietin receptor (EPOR): EPOR is the receptor for erythropoietin. It is expressed primarily in bone marrow, but is also expressed in other tissues, including kidney, thyroid, adrenal, and lung. Binding of EPO to EPOR appears to stimulate erythroid proliferation and/or survival. It also has neuroprotective effects in vitro and in models of neurodegenerative diseases. Exemplary human EPOR sequences include GenBank Accession No. NM_000121 (nucleic acid) and NP_000112 (amino acid). However, one of ordinary skill in the art can identify additional EPOR sequences.

“Erythropoietin receptor agonists” refers to a group of compounds that bind to and activate the erythropoietin receptor. EPO is the endogenous erythropoietin receptor agonist. Erythropoietin receptor agonists also include asialo-erythropoietin and carbamoylated erythropoietin (CEPO), ARA 290, which have non-erthryopoietic activities. Additional EPO receptor agonists include darbepoetin alfa (ARANESP®), Epoetin alfa (EPOGEN®, PROCRIT®, or RETACRIT®), and methoxy polyethylene glycol-Epoetin beta (MIRCERA®).

Heterologous: Originating from a different genetic source. A nucleic acid molecule or protein that is heterologous to a cell originates from a genetic source other than the cell in which it is expressed, or is not normally expressed in the cell. In one specific, non-limiting example, a heterologous nucleic acid molecule encoding a protein, such as a thrombopoietin receptor or an erythropoietin receptor, is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

Isolated: A biological component (such as a nucleic acid or protein, or cell) that has been substantially separated, produced apart from, or purified away from other biological components, for example other nucleic acids, proteins, and/or cells. Thus, isolated nucleic acids and proteins include nucleic acids and proteins purified by standard purification methods. The term also embraces nucleic acids and proteins prepared by recombinant expression in a host cell, as well as chemically synthesized nucleic acids. An isolated nucleic acid, protein, or cell can be at least 50%, at least 60%, at least 70%, at least 80%, at least 90%, at least 95%, at least 96%, at least 97%, at least 98%, or at least 99% pure.

Natural Killer (NK) cells: Cells of the immune system that kill target cells in the absence of a specific antigenic stimulus and without restriction according to MHC class. Target cells can be tumor cells or cells harboring viruses. NK cells are characterized by the presence of CD56 and the absence of CD3 surface markers. NK cells typically comprise approximately 10 to 15% of the mononuclear cell fraction in normal peripheral blood. Historically, NK cells were first identified by their ability to lyse certain tumor cells without prior immunization or activation. NK cells are thought to provide a “back up” protective mechanism against viruses and tumors that might escape the CTL response by down-regulating MHC class I presentation. In addition to being involved in direct cytotoxic killing, NK cells also serve a role in cytokine production, which can be important to control cancer and infection.

In some examples, a “modified NK cell” is a NK cell transduced with a heterologous nucleic acid (such as one or more of the nucleic acids or vectors disclosed herein) or expressing one or more heterologous proteins. The terms “modified NK cell” and “transduced NK cell” are used interchangeably in some examples herein.

Pharmaceutically acceptable carriers: The pharmaceutically acceptable carriers of use are known to those of ordinary skill in the art. Remington: The Science and Practice of Pharmacy, 22^{nd} ed., London, UK: Pharmaceutical Press, 2013, describes compositions and formulations suitable for pharmaceutical delivery of the disclosed agents. In general, the nature of the carrier will depend on the particular mode of administration being employed. For example, parenteral formulations usually include injectable fluids that include pharmaceutically and physiologically acceptable fluids such as water, physiological saline, balanced salt solutions, aqueous dextrose, glycerol or the like as a vehicle.

In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some embodiments, the active agent and pharmaceutically acceptable carrier are provided in a unit dosage form such as in a selected quantity in a vial. Unit dosage forms can include one dosage or multiple dosages (for example, in a vial from which metered dosages of the agent can selectively be dispensed).

Purified: The term purified does not require absolute purity; rather, it is intended as a relative term. Thus, for example, a purified protein or cell preparation is one in which the protein or cell is more enriched than in its original environment. In one embodiment, a preparation is purified such that the protein or cells represent at least 50% of the total peptide or protein content of the preparation.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the manipulation of isolated segments of nucleic acids, for example, by genetic engineering techniques. In several embodiments, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid, or the nucleic acid can be integrated into the host cell chromosome.

Subject: Living multi-cellular vertebrate organisms, a category that includes human and non-human mammals, such as non-human primates, pigs, sheep, cows, dogs, cats, rodents, and the like. In an example, a subject is a human.

T cells: A white blood cell (lymphocyte) that is an important mediator of the immune response. T cells include, but are not limited to, CD4⁺ T cells and CD8⁺ T cells. A CD4⁺ T cell is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, a CD8⁺ T cell is a cytotoxic T lymphocyte (CTL). In another embodiment, a CD8⁺ cell is a suppressor T cell.

Activated T cells can be detected by an increase in cell proliferation and/or expression of or secretion of one or more cytokines (such as IL-2, IL-4, IL-6, IFNγ, or TNFα). Activation of CD8+ T cells can also be detected by an increase in cytolytic activity in response to an antigen.

A natural killer T (NK-T) cell is a class of T cell that expresses both T-cell receptors (TCR) characteristic of adaptive immunity, and surface receptors for NK cells (e.g. the cell-surface marker NK1.1 normally associated with NK cells along with an α/β T-cell receptor). See, e.g., Tupin et al., Nat Rev Microbiol 5:405-417, 2007.

In some examples, a “modified T cell” is a T cell transduced with a heterologous nucleic acid (such as one or more of the nucleic acids or vectors disclosed herein) or expressing one or more heterologous proteins. The terms “modified T cell” and “transduced T cell” are used interchangeably in some examples herein. Similarly, a “modified NK-T cell” is an NK-T cell transduced or transfected with a heterologous nucleic acid (such as one or more of the nucleic acids or vectors disclosed herein) or expressing one or more heterologous proteins.

Thrombopoietin (TPO): Thrombopoietin is a hematopoietic growth factor that promotes megakaryocyte proliferation and maturation and platelet production (thrombopoiesis). TPO is primarily produced by the liver and kidneys Exemplary human TPO sequences include GenBank Accession No. NM_000460 (nucleic acid) and NP_000451 (amino acid). However, one of ordinary skill in the art can identify additional TPO sequences.

“Thrombopoietin receptor agonists” refers to a group of compounds that bind to and activate the thrombopoietin receptor. TPO is the endogenous thrombopoietin receptor agonist. The peptide romiplostim is also a thrombopoietin receptor agonist. Two small molecule thrombopoietin receptor agonists are eltrombopag and avatrombopag. Romiplostim, eltrombopag, and avatrombopag are all used clinically in the treatment of thrombocytopenias.

Thrombopoietin receptor (c-MPL): c-MPL is the receptor for thrombopoietin. Binding of TPO to c-MPL appears to stimulate megakaryocyte proliferation and platelet formation. Exemplary human c-MPL sequences include GenBank Accession No. NM_005373 (nucleic acid) and NP_005364 (amino acid). However, one of ordinary skill in the art can identify additional c-MPL sequences.

Transduced or transformed: A transformed cell is a cell into which a nucleic acid molecule has been introduced by molecular biology techniques. As used herein, the term transformed and the like (e.g., transformation, transfection, transduction, etc.) encompasses all techniques by which a nucleic acid molecule might be introduced into such a cell, including transduction with viral vectors, transformation with plasmid vectors, and introduction of DNA by electroporation, lipofection, and particle gun acceleration.

Treating or inhibiting a disorder: “Inhibiting” a condition refers to inhibiting the full development of a condition or disease, for example, cancer or a tumor. Inhibition of a condition can span the spectrum from partial inhibition to substantially complete inhibition of the disease. In some examples, the term “inhibiting” refers to reducing or delaying the onset or progression of a condition. “Treatment” refers to a therapeutic intervention that ameliorates a sign or symptom of a disease or condition after it has begun to develop. A subject to be administered an effective amount of the disclosed T cells or NK cells can be identified by standard diagnosing techniques for such a disorder, for example, presence of the disease or disorder or risk factors to develop the disease or disorder.

Vector: A nucleic acid molecule (such as a DNA or RNA molecule) including a promoter(s) that is operably linked to the coding sequence of a protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. In some embodiments, a vector includes a nucleic acid molecule encoding a thrombopoietin receptor or an erythropoietin receptor. In some examples, the vector is a bacterial vector. In other examples, the vector is a viral vector, such as a nucleic acid vector having at least some nucleic acid sequences derived from one or more viruses. In some embodiments, the viral vector is a lentiviral vector.

### II. Modified Immune Cells

Provided herein are modified immune cells that express a heterologous hematopoietic growth factor receptor, for example, a heterologous thrombopoietin receptor (c-MPL) or a heterologous erythropoietin receptor (EPOR). In some embodiments, the modified immune cells are NK cells, T cells, or NK-T cells. In further embodiments, the modified immune cells are NK cells, T cells, or NK-T cells that also express a chimeric antigen receptor (CAR), such as CAR-NK cells, CAR-T cells, or CAR-NK-T cells.

In some embodiments, modified immune cells (e.g., NK cells, T cells, NK-T cells, CAR-NK cells, CAR-T cells, or CAR-NK-T cells) expressing a heterologous thrombopoietin receptor (c-MPL) are provided. In particular non-limiting examples, modified NK cells expressing a heterologous c-MPL are provided. In some examples, the heterologous thrombopoietin receptor expressed in the modified cells has at least 95% sequence identity (such as at least 96%, at least 97%, at least 98%, or at least 99% sequence identity) to the amino acid sequence of SEQ ID NO: 1. In other examples, the heterologous thrombopoietin receptor expressed in the modified cells includes or consists of the amino acid sequence of SEQ ID NO: 1. In other examples, the nucleic acid encoding the heterologous thrombopoietin receptor has at least 95% sequence identity (such as at least 96%, at least 97%, at least 98%, or at least 99% sequence identity) to the nucleic acid sequence of SEQ ID NO: 2. In other examples, the heterologous thrombopoietin receptor expressed in the modified cells is encoded by a nucleic acid that includes or consists of the nucleic acid sequence of SEQ ID NO: 2.

In other embodiments, modified immune cells (e.g., NK cells, T cells, NK-T cells, CAR-NK cells, CAR-T cells, or CAR-NK-T cells) expressing a heterologous erythropoietin receptor (EPOR) are provided. In particular non-limiting examples, modified NK cells or modified T cells expressing a heterologous EPOR are provided. In some examples, the heterologous EPOR expressed in the modified cells has at least 95% sequence identity (such as at least 96%, at least 97%, at least 98%, or at least 99% sequence identity) to the amino acid sequence of SEQ ID NO: 3. In other examples, the heterologous EPOR expressed in the modified cells includes or consists of the amino acid sequence of SEQ ID NO: 3. In other examples, the nucleic acid encoding the heterologous EPOR has at least 95% sequence identity (such as at least 96%, at least 97%, at least 98%, or at least 99% sequence identity) to the nucleic acid sequence of SEQ ID NO: 4. In other examples, the heterologous EPOR expressed in the modified cells is encoded by a nucleic acid that includes or consists of the nucleic acid sequence of SEQ ID NO: 4.

In some examples, the immune cells (such as T cells or NK cells) are obtained from peripheral blood (such as peripheral blood mononuclear cells), lymph node, thymus, bone marrow, tumor tissue, adipose tissue, human embryonic stem cells (hESC), induced pluripotent stem cells (iPSC), or umbilical cord blood. In some embodiments, the cells are autologous to a subject being treated. In other examples, the cells are allogeneic to the subject.

In some embodiments, the immune cells (e.g., T cells or NK cells) are transduced with a vector including a heterologous nucleic acid encoding a thrombopoietin receptor or erythropoietin receptor, including, but not limited to a vector including SEQ ID NO: 2 or SEQ ID NO: 4. In some non-limiting examples, the vector includes or consists of the nucleic acid sequence of SEQ ID NO: 5 or SEQ ID NO: 6. In some examples, the vector is a viral vector, such as a lentiviral vector.

Viral vectors suitable for gene delivery to NK cells or T cells include retrovirus, adenovirus, adeno-associated virus, vaccinia virus, fowlpox, and lentivirus vectors. In particular non-limiting examples disclosed herein, NK cells or T cells are transduced with lentiviral vectors including a heterologous nucleic acid encoding a thrombopoietin receptor or erythropoietin receptor. Some advantages of using a lentiviral system include long-term expression of the transgene, the ability to transduce both dividing cells and non-dividing cells, the ability to deliver complex genetic elements, lack of expression of viral proteins after transduction, lack of insertional mutagenesis in human cells, high titer production, and ease of vector manipulation and production.

In particular examples, the nucleic acid encoding the thrombopoietin receptor or erythropoietin receptor is included in a lentiviral gene transfer vector. The receptor nucleic acid in the transfer vector is operably linked to one or more expression control elements, such as a promoter. Exemplary promoters include constitutive promoters such as cytomegalovirus (CMV), SV40, phosphoglycerate kinase (PGK), ubiquitin C (UBC), elongation factor-1 (EFS), chicken β-actin short promoter (CBH), EF-1 alpha (EF1a) promoter, or EF1a short promoter, a hybrid promoter (such as a CMV enhancer fused to chicken β-actin promoter (CAG)), or an inducible or tissue-specific promoter.

Additional expression control elements that may be included in the transfer vector include sequences that control or regulate transcription and/or translation of a nucleic acid, such as enhancers, leader sequences, transcription terminators, start and/or stop codons, internal ribosome entry sites (IRES), splicing signals, and polyadenylation signals. In examples where the vector or construct includes two (or more) heterologous nucleic acids of interest, the nucleic acids are operably linked, for example, separated by an IRES or other multicistronic element such as a P2A and/or T2A element. The vector may also contain additional elements such as packaging signals (e.g., lentivirus w packaging signal), a central polypurine tract (cPPT), a woodchuck hepatitis virus post-transcriptional regulatory element (WPRE), and/or a Rev Response element (RRE). In some examples, the lentivirus vector is self-inactivating.

Lentivirus vectors including one or more nucleic acids of interest can be prepared by one of ordinary skill in the art utilizing conventional molecular biology techniques. For example, the nucleic acid of interest can be cloned into a lentivirus transfer vector. Lentivirus plasmid systems (such as 3 or 4 plasmid systems) are commercially available, for example from Clontech (Mountain View, Calif.), ThermoFisher Scientific (Waltham, Mass.), or Addgene (Cambridge, Mass.).

In some embodiments, modified (e.g., transduced) T cells expressing an EPOR or c-MPL are produced by obtaining a population of lymphocytes (such as a population of PBMCs) from a subject, for example by apheresis. Naïve or quiescent T cells in the population of lymphocytes are activated prior to transduction, for example, by contacting the lymphocytes with one or more cytokines (such as one or more of IL-2, IL-4, IL-6, IL-7, IL-12, IL-15, and IL-23). In some examples, the lymphocytes are contacted with anti-CD3 antibody and IL-2 for 1-4 days (such as 1 day, about 2 days, about 3 days, or about 4 days) to produce activated T cells. In some examples, the lymphocytes are contacted with 30 ng/ml anti-CD3 antibody and 300 IU/ml IL-2 for 2 or 3 days.

The activated T cells are transduced or transfected with a vector encoding an EPOR or c-MPL protein. In some examples, the transduced T cells are enriched and/or expanded. The transduced T cells can be expanded by culturing the transduced T cells with anti-CD3 (e.g., about 30 ng/ml), anti-CD28 (e.g., about 30 ng/ml), and/or IL-2 (e.g., about 300 IU/ml) for a period of time (such as about 7-14 days or 9-11 days). In some examples, the transduced T cells are expanded by culture on irradiated PBMC cells.

In other embodiments, modified (e.g., transduced) NK cells expressing an EPOR or c-MPL are produced by obtaining a population of lymphocytes (such as a population of PBMCs) from a subject, for example by apheresis. In some examples, the NK cells are activated by culturing isolated NK cells with one or more cytokines for a period of time prior to transduction. In some examples, the NK cells are cultured in culture medium including IL-2, IL-15, and/or IL21 for 1-14 days prior to transduction. In some non-limiting examples, the NK cells are activated in culture medium including 500 IU/ml IL-2 or 1000 IU/ml IL-2 for 1-6 days, 2-3 days, or 3-5 days.

The activated NK cells are transduced or transfected with a vector encoding an EPOR or c-MPL protein. The transduced NK cells can also be enriched and/or expanded, for example by culturing the transduced NK cells in a medium including IL-2 and/or one or more additional cytokines. In some examples, the NK cells are expanded on feeder cells, such as EBV-LCLs (TM-LCL, SMI-LCL), allogeneic or autologous PBMCs, Wilms tumor cell line HFWT, and K562 cells (such as genetically modified K562 cells, for example, K562-mb15-41BBL or K562-mbIL-21 cells.

In some embodiments of the disclosed methods, the transduced NK cells or T cells are cultured with a thrombopoietin receptor agonist (if the modified cells express c-MPL) or an erythropoietin receptor agonist (if the modified cells express EPOR) for example, prior to administering to a subject. In some examples, the transduced cells are cultured with about 10-250 ng/ml of a thrombopoietin receptor agonist (e.g., about 10-50 ng/ml, 25-75 ng/ml, 50-100 ng/ml, 75-150 ng/ml, 125-175 ng/ml, 150-200 ng/ml, 175-225 ng/ml, or 200-250 ng/ml). In one particular example, the transduced cells are cultured with about 50 ng/ml TPO. In other examples, the transduced cells are cultured with about 1-50 U/ml of an EPOR agonist (e.g., about 1-5 U/ml, about 2.5-7.5 U/ml, about 5-10 U/ml, about 7.5-15 U/ml, about 10-20 U/ml, about 15-25 U/ml, about 25-40 U/ml, or about 35-50 U/ml). In a particular example, the transduced cells are cultured with about 5 U/ml EPO. The transduced cells are cultured with the thrombopoietin receptor agonist or erythropoietin receptor agonist for about 7-28 days (e.g., about 7-10 days, about 8-12 days, about 10-15 days, about 12-18 days, about 14-21 days, about 16-24 days, or about 21-28 days), for example, prior to cryopreserving or administering the cells to a subject. In particular examples, the transduced cells are cultured with the thrombopoietin receptor agonist or erythropoietin receptor agonist for about 10-17 days (such as about 10, 11, 12, 13, 14, 15, 16, or 17 days).

In some embodiments, the transduced NK cells or T cells are cultured with at least one cytokine in addition to the thrombopoietin receptor agonist or erythropoietin receptor agonist. In some examples, the transduced NK cells or T cells are cultured with IL-2 in addition to the thrombopoietin receptor agonist or erythropoietin agonist. In particular examples the amount of IL-2 is a reduced amount of IL-2 (“low dose”), which in some examples, is about 1-50 U/ml IL-2 (for example, about 1-5 U/ml, about 2-10 U/ml, about 5-15 U/ml, about 10-25 U/ml, about 20-40 U/ml, about 25-50 U/ml, or about 1-25 U/ml).

### III. Methods of Treating Cancer

Methods of treating cancer with the modified immune cells disclosed herein are provided. In some embodiments, the methods include administering modified immune cells (e.g., modified NK cells, T cells, NK-T cells, CAR-NK cells, CAR-T cells, or CAR-NK-T cells) expressing a hematopoietic growth factor receptor (such as a thrombopoietin receptor or an erythropoietin receptor) to a subject with cancer. In some embodiments, the subject is also administered an agonist for the hematopoietic growth factor receptor expressed by the modified cells (e.g., a thrombopoietin receptor agonist or erythropoietin receptor agonist).

In some embodiments, the subject has a hematological malignancy. In some examples, the subject is administered modified NK cells or T cells expressing c-MPL or EPOR. In other examples, the subject is administered modified CAR-NK cells or CAR-T cells expressing c-MPL or EPOR and a CAR targeting the hematological malignancy. Examples of hematological malignancies include leukemias, including acute leukemias (such as 11q23-positive acute leukemia, acute lymphocytic leukemia (ALL), T-cell ALL, acute myelocytic leukemia, acute myelogenous leukemia (AML), and myeloblastic, promyelocytic, myelomonocytic, monocytic and erythroleukemia), chronic leukemias (such as chronic myelocytic (granulocytic) leukemia, chronic myelogenous leukemia (CML), and chronic lymphocytic leukemia (CLL)), lymphoblastic leukemia, polycythemia vera, lymphoma, diffuse large B cell lymphoma, Burkitt lymphoma, T cell lymphoma, follicular lymphoma, mantle cell lymphoma, Hodgkin disease, non-Hodgkin lymphoma, multiple myeloma, Waldenstrom macroglobulinemia, heavy chain disease, myelodysplastic syndrome (MDS), hairy cell leukemia, and myelodysplasia. In particular examples, the subject has a leukemia or lymphoma, such as AML, ALL, CLL, multiple myeloma, MDS, a B cell lymphoma, or a T cell lymphoma.

In other examples, the subject has a solid tumor. In some examples, the subject is administered modified NK cells or T cells expressing c-MPL or EPOR. In other examples, the subject is administered modified CAR-NK cells or CAR-T cells expressing c-MPL or EPOR and a CAR targeting the solid tumor. Examples of solid tumors, include sarcomas (such as fibrosarcoma, myxosarcoma, liposarcoma, chondrosarcoma, osteogenic sarcoma, and other sarcomas), synovioma, mesothelioma, Ewing sarcoma, leiomyosarcoma, rhabdomyosarcoma, colon cancer, colorectal cancer, peritoneal cancer, esophageal cancer, pancreatic cancer, breast cancer (including basal breast carcinoma, ductal carcinoma and lobular breast carcinoma), lung cancer, ovarian cancer, prostate cancer, liver cancer (including hepatocellular carcinoma), gastric cancer, squamous cell carcinoma (including head and neck squamous cell carcinoma), basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, hepatoma, bile duct carcinoma, choriocarcinoma, Wilms tumor, cervical cancer, fallopian tube cancer, testicular tumor, seminoma, bladder cancer, kidney cancer (such as renal cell cancer), melanoma, and CNS tumors (such as a glioma, glioblastoma, astrocytoma, medulloblastoma, craniopharyrgioma, ependymoma, pinealoma, hemangioblastoma, acoustic neuroma, oligodendroglioma, meningioma, neuroblastoma and retinoblastoma). Solid tumors also include tumor metastases (for example, metastases to the lung, liver, brain, or bone). In particular examples, the subject has renal cell cancer, breast cancer, colon cancer, bladder cancer, or a uroepithelia carcinoma.

The modified immune cells (such as T cells, NK cells, NK-T cells, CAR-T cells, CAR-NK cells, or CAR-NK-T cells) expressing a thrombopoietin receptor or a erythropoietin receptor described herein can be incorporated into pharmaceutical compositions. Such compositions typically include a population of cells and a pharmaceutically acceptable carrier. A “pharmaceutically acceptable carrier” includes any and all solvents, dispersion media, coatings, antibacterial and antifungal agents, isotonic and absorption delaying agents, and the like, compatible with pharmaceutical administration (see, e.g., Remington: The Science and Practice of Pharmacy, 22^{nd} ed., London, UK: Pharmaceutical Press, 2013). Examples of such carriers or diluents include, but are not limited to, water, saline, Ringer's solutions, dextrose solution, balanced salt solutions, and 5% human serum albumin. Liposomes and non-aqueous vehicles such as fixed oils may also be used. Supplementary active compounds can also be incorporated into the compositions. Actual methods for preparing administrable compositions include those provided in Remington: The Science and Practice of Pharmacy, 22^{nd} ed., London, UK: Pharmaceutical Press, 2013.

In some examples, the composition includes about 10⁴ to 10¹² of the modified immune cells (for example, about 10⁴-10⁸ cells, about 10⁶-10⁸ cells, or about 10⁶-10¹² cells). For example, the composition may be prepared such that about 10⁴ to 10¹⁰ modified NK cells or modified T cells cells/kg (such as about 10⁴, 10⁵, 10⁶, 10⁷, 10⁸, 10⁹, or 10¹⁰ cells/kg) are administered to a subject. In specific examples, the composition includes at least 10⁴, 10⁵, 10⁶, or 10⁷ modified immune cells (e.g., modified NK cells, T cells, NK-T cells, CAR-NK cells, CAR-T cells, or CAR-NK-T cells). The population of modified cells is typically administered parenterally, for example intravenously; however, other routes of administration can also be used. Appropriate routes of administration can be determined based on factors such as the subject, the condition being treated, and other factors.

Multiple doses of the population of modified immune cells can be administered to a subject. For example, the cells can be administered daily, every other day, twice per week, weekly, every other week, every three weeks, monthly, or less frequently. A skilled clinician can select an administration schedule based on the subject, the condition being treated, the previous treatment history, and other factors.

In particular embodiments, the subject is also administered a thrombopoietin agonist, if the modified cells express a thrombopoietin receptor. In particular examples, the thrombopoietin receptor agonist is thrombopoietin. In other examples, the thrombopoietin receptor agonist is romiplostim, eltrombopag, or avatrombopag. In some examples, the subject is administered 0.3-2.4 μg/kg of TPO, about 1-10 μg/kg of romiplostim per week, about 25-150 mg of eltrombopag per day, or about 20-100 mg of avatrombopag per day. However, one of ordinary skill in the art will recognize that these dosages and frequencies can be varied, for example, as a result of clinical trials, the particular subject being treated, or other factors. In some examples, the thrombopoietin receptor agonist is administered to the subject prior to, substantially simultaneously with (e.g., at approximately the same time), or subsequent to administration of the modified cells. In particular examples, the thrombopoietin receptor agonist is administered to the subject for up to 6 months, or in other examples for about 7-30 days.

In other embodiments, the subject is also administered an erythropoietin receptor agonist, if the modified cells express an erythropoietin receptor. In particular examples, the erythropoietin receptor agonist is erythropoietin. In other examples, the erythropoietin receptor agonist is asialo-erythropoietin, carbamoylated erythropoietin (CEPO), or ARA 290. In still further examples, the EPO receptor agonist is darbepoetin alfa (ARANESP®), epoetin alfa (EPOGEN®, PROCRIT®, or RETACRIT®), or methoxy polyethylene glycol-epoetin beta (MIRCERA®). In some examples, the subject is administered 2-4 mg of ARA 290, about 0.45 μg/kg to 5 mg/kg of darbepoietin alfa, about 50-600 U/kg of epoietin alfa, or 0.6 μg/kg or more of epoietin beta. However, one of ordinary skill in the art will recognize that these dosages can be varied, for example, as a result of clinical trials, the particular subject being treated, or other factors. In some examples, the erythropoietin receptor agonist is administered to the subject prior to, substantially simultaneously with (e.g., at approximately the same time), or subsequent to administration of the modified cells. In particular examples, the erythropoietin receptor agonist is administered to the subject for up to 6 months, or in other examples for about 7-30 days.

In additional examples, the subject is also administered at least one cytokine (such as IL-2). In some examples, the cytokine supports survival and/or proliferation of the modified cells. In specific, non-limiting examples, the subject is administered “low dose” IL-2. In some examples, low dose IL-2 is about 0.5-4 million units/m² (e.g., about 0.5-1 million units/m², about 0.75-1.25 million units/m², about 1-1.5 million units/m², about 1.25-1.75 million units/m², about 1.5-2 million units/m², about 2.25-2.75 million units/m², about 2.5-3 million units/m², about 3-3.5 million units/m², about 3.25-3.75 million units/m², or about 3.5-4 million units/m². The IL-2 may be administered before, after, or substantially simultaneously (e.g., at approximately the same time) with the modified cells. In some non-limiting examples, IL-2 is administered one or more times prior to or substantially simultaneously with administration of the modified cells. In particular examples, IL-2 is administered to the subject once or twice daily for 2-14 days, such as 2, 3, 4, 5, 6, 7, 8, 9, 10, 11, 12, 13, or 14 days. In other examples, the subject is administered IL-2 for about 7 days.

In some examples, the subject is also treated with one or more of surgery, radiation therapy, chemotherapeutic agents, monoclonal antibodies, or other therapies. A skilled clinician can select appropriate additional therapies for the subject, depending on factors such as the subject, the cancer being treated, treatment history, and other factors.

### EXAMPLES

The following examples are provided to illustrate certain particular features and/or embodiments. These examples should not be construed to limit the disclosure to the particular features or embodiments described.

### Example 1

### Materials and Methods

Cell culture and cell lines: NK-92, Raji, 721.221, and K562 cell lines were purchased from ATCC (VA, USA). The KHYG-1 cell line was purchased from Leibniz Institute DSMZ (Germany). NK-92 cells were cultured in RPMI medium supplemented with 12.5% heat-inactivated fetal bovine serum (FBS), 12.5% horse serum and 2 mM L-glutamine (Life Technologies). The KHYG-1 cells were cultured in RPMI medium containing 10% FBS, penicillin/streptomycin (100 μg/ml) and 2 mM L-glutamine. Both NK-92 and KHYG-1 were supplemented with 200 U/ml recombinant human (rh) IL-2 (Roche). Raji, K562, and 721.221 cells were also cultured in RPMI medium with 10% FBS, while 293T cells were maintained in Dulbecco's Modified Eagle's medium (DMEM) with 10% FBS and 2 mM L-glutamine. All cell cultures were at 37° C. with 5-6.5% CO₂.

Lentiviral production and transduction: Lentivirus gene expression vectors encoding EGFP-P2A linker-human EPOR or EGFP-P2A-human c-MPL were cloned according to our design by VectorBuilder (TX, USA). Plasmid DNA for the above and pCMV-dR8.2 dvpr and pCMV-VSV-G packaging plasmids was purified using ZymoPUREII-EndoZero plasmid maxiprep kit (Zymo Research, CA). Lentiviral particles were generated with HEK293T cells and Lipofectamine3000 reagent (Thermo Fisher Scientific, MA) following the manufacturers' instructions. Viral supernatant was harvested at 24 and 48 hours, pooled, and concentrated with lenti-X concentrator (Takara Bio, Japan) or by centrifugation at 18,600 rpm for 2 hours at 4° C. The target cells were transduced with lentivirus at a multiplicity of infection (MOI) of 20 using retronectin (Takara Bio, Japan) coated plate protocol. After 5 days post-transduction, cells were stained and sorted with fluorescence-activated cell sorting (FACS) using a BD FACSAria II to purify GFP⁺EPOR⁺ and GFP⁺c-MPL⁺ NK cell populations.

In vitro cell expansion cultures: The NK-92 (5×10⁴ cell/ml) and KHYG-1 (1×10⁴ cell/ml) cells were cultured in 1 ml of complete medium in 24-well plate. The NK cell lines were supplemented with different IL-2 doses, 5 U/ml rhEPO (R&D) and/or 50 ng/ml rhTPO (R&D) depending on experiments. Trypan blue negative cells were counted every other day. On day 4, the medium was changed and replaced with fresh IL-2/EPO/TPO as their initial conditions. The short-term cell expansion assay was performed in similar fashion, except cells were initially cultured without IL-2 overnight before adding different cytokines and enumerating the cell number. For competitive primary NK and T cell expansion assay, all transduced cells (5×10⁵/ml for NK and 4×10⁵/ml for T cell) containing a mixture of GFP positive (transduced) and GFP negative (untransduced) cells were plated on day 0. Fresh medium with appropriate supplements was replaced every other day for NK cells and only on day 3 for T cell experiment. Viable cells were counted and percentage of GFP⁺ cells was acquired on indicated days (NK: day 4 and 6, T: day 3 and 6). The fold change of GFP⁺ cells was calculated by multiplying the counted cell number times the fraction GFP⁺.

Flow cytometry, reagents and pathway analysis: Fluorochrome-conjugated monoclonal antibodies were used as follows: BD Biosciences: CD56-PE-Cy7 (NCAM16.2), CD3-PE (UCHT1), c-MPL-PE (1.6.1), STAT5(pY694)-Alexa Fluor647 (47/stat5(pY694)), NKp46-APC (9E2), NKp44-PE (p44-8), FAS-PE (DX2) CD62L-V450 (DREG-56); R&D systems: EPOR-PE (FAB307P); Biolegend: Bcl-2-PE (100), TRAIL-APC (RIK-2), CXCR4-BV421 (12G5), KIR3DL1-BV421 (DX9); Abcam: Bcl-xL-PE (ab208747); Cell signaling technology: P-p44/42 MAPK-Alexa Fluor647 (E10) and p-S6 ribosomal protein-PE (D57.2.2E); Beckman Coulter: NKG2A-PE (Z199), KIR2DL1/S1-PE (EB6B), KIR2DL2/3-PE/Cy5.5 (GL183). Staining was performed in phosphate-buffered saline (PBS) with 2% FBS and 2 μM EDTA and analyzed on a BD LSRFortessa machine. If intracellular staining was required, BD cytofix/cytoperm (BD Biosciences) was applied for cell fixation and permeabilization after surface staining. The apoptosis assay was performed using PE annexinV apoptosis detection kit (BD Biosciences). The data were analyzed on FlowJo software.

For measurement of cell signaling and anti-apoptotic proteins, cells were cultured overnight without IL-2 then incubated in appropriate medium supplemented with different IL-2/EPO/TPO conditions for 15 minutes for phospho-protein detection and 24 hours for anti-apoptotic protein studies. Expression of target proteins was evaluated afterward by flow cytometry as described above.

Flow cytometry based killing assay: The effector cells were prepared by culturing EPOR⁺ NK-92 and c-MPL⁺ NK-92 cells in different IL-2/EPO/TPO concentrations for 24-40 hours before the experiment. The target cells were labeled with 1 μM cell trace violet in some experiments (Thermo Fisher Scientific). The effector and target cells were incubated together in duplicate wells at specific E:T ratio in complete medium without cytokines for 4 hours (total cell number: 200,000 to 250,000 per well in 96-well plate). 7-AAD and Sphero AccuCount Fluorescent particles (Spherotech, IL) were added before analysis by flow cytometry. The absolute number of viable 7-AAD negative target cells was calculated according to bead numbers added (Cooper et al., Leukemia 32:1970-1983, 2018). The percentage of dead cells was calculated relative to target cell only control wells.

Primary NK cell activation and transduction: Peripheral blood NK cells and T cells from healthy volunteers were isolated from de-identified buffy-coat samples. NK cells were separated by immune-density negative selection and density gradient centrifugation using RosetteSep human NK cell enrichment cocktail (Stemcell Technologies, Canada) and lymphocyte separation medium (MP Biomedicals, CA). The primary NK cells were activated with irradiated Epstein-Barr virus (EBV) transformed lymphoblastic cell line (SMI-LCL) (Berg et al., Cytotherapy 11:341-355, 2009) and maintained in X-Vivo20 medium with 10% heat-inactivated human serum, 2 mM L-glutamine and 500 U/ml IL-2. After 4-5 days of NK cell activation, NK cells were transduced with the previously described lentiviral particles (MOI=20) in combination with 1.5 μM BX795 (InvivoGen, CA). On day 3 post-transduction, cells were evaluated for their expression of CD3, GFP, CD56, EPOR and c-MPL expression. The transduction efficiency (measured as GFP positive NK cells) was 4-57%. Both activated GFP⁺ and GFP⁻ NK cells were utilized in the competitive cell expansion assay.

Degranulation assay and cytokine production: Transduced human NK cells or control untransduced cells were cultured with IL-2 and/or TPO for 4 days, then mixed at a 1:1 ratio with K562 target cells (2.5×10⁵ total cells per well of 96 well plate) in medium lacking added cytokines. To examine degranulation, cells were washed after two hours and stained with anti-CD107a (H4A3) (BioLegend). To assay cytokine production, GolgiPlug and GolgiStop (BD Biosciences) were added at manufacturer's recommended concentrations within the first hour of co-culture with K562. After 4 hours, cells were washed and stained for cell surface markers, then stained intracellularly with mAb recognizing TNF-α (Mab 11) and IFN-γ (B27) (BD Biosciences).

Persistence of transduced human NK cells in immunodeficient mice: NK cells from human PBMC were transduced with matched lentiviral vectors encoding GFP/MPL or GFP/CD34 then mixed at a 1:1 ratio and injected intravenously into NSG mice (2.2×107 total cells/mouse). Mice received daily intraperitoneal injections of human TPO (50 μg/kg/mouse) or human IL-2 (10,000 IU/mouse) for 4 days. After euthanasia, blood was drawn by cardiac puncture, bone marrow was harvested by flushing the tibia, and spleen, liver, and lung fragments were processed by grinding over 70 μm nylon mesh. Blood, bone marrow, lung, and spleen samples received 1-2 rounds of 5 minute incubations with ACK lysis buffer (Quality biological). Liver samples were purified by centrifugation over 35% Percoll (Sigma) solution in RPMI. The presence of transduced human cells was quantified in various organs via flow cytometry using antibodies to human MHC class I (W6/32) (BioLegend), c-MPL (1.6.1), CD34 (581) (BD BioSciences), GFP-fluorescence, and Live/Dead Aqua viability dye (Thermo Fisher).

Primary T cell activation and transduction: Peripheral blood mononuclear cells (PBMC) were obtained from healthy donor buffy-coat samples as specified previously and were separated by density gradient centrifugation. The PBMC were activated with the CD3 antibody (OKT3, Invitrogen)-coated plate and soluble CD28 antibody (CD28.2, Biolegend) and maintained in RPMI with 10% FBS and 300 U/ml IL-2. After 2 days of T cell activation, cells were transduced with the previously described lentiviral particles (MOI=10). On day 4 post-transduction, cells were evaluated for the expression of CD3, GFP, EPOR and c-MPL expression, in each case showing >95% CD3⁺ cells. The transduction efficiency (measured as GFP positive T cells) was 40-60%. Both activated GFP⁺ and GFP⁻ T cells were utilized in the competitive cell expansion assay.

Statistical analysis: Data were analyzed and presented using GraphPad Prism software. The mean, standard deviation (SD) and p-value were displayed with statistical tests as indicated.

### Example 2

### Exogenous Expression of EPOR or c-MPL in Human NK Cell Lines Enhances their Proliferative Response to Low-Dose IL-2 in the Presence of EPO or TPO

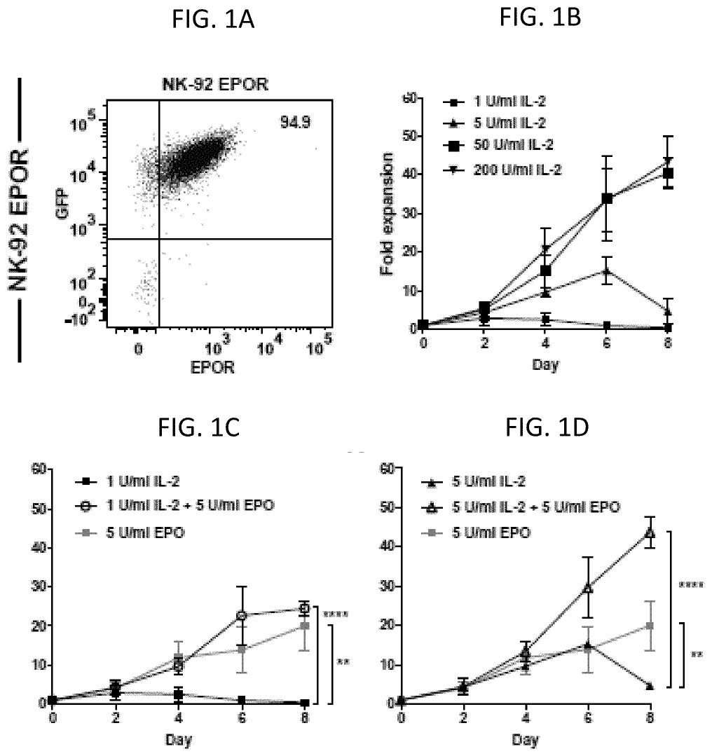

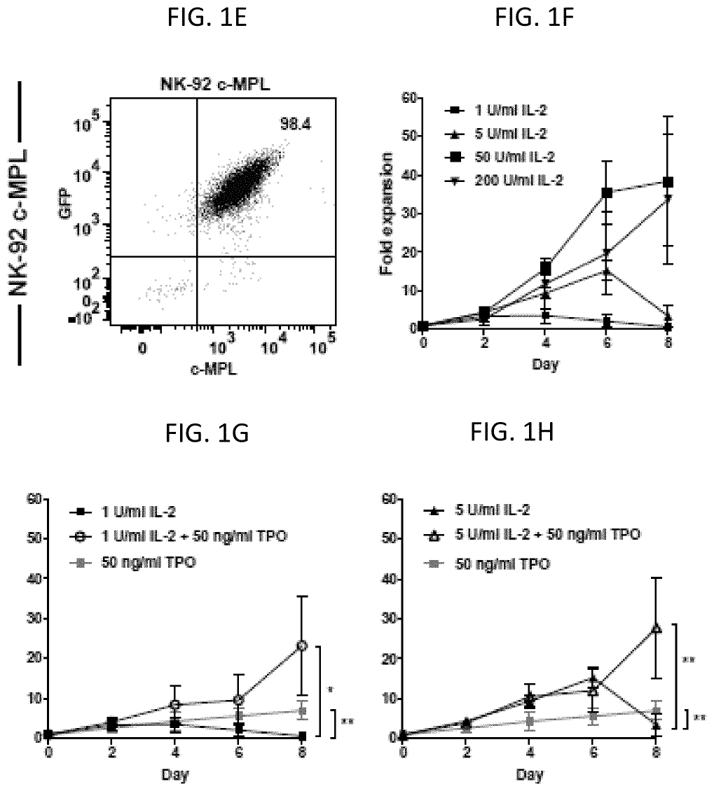

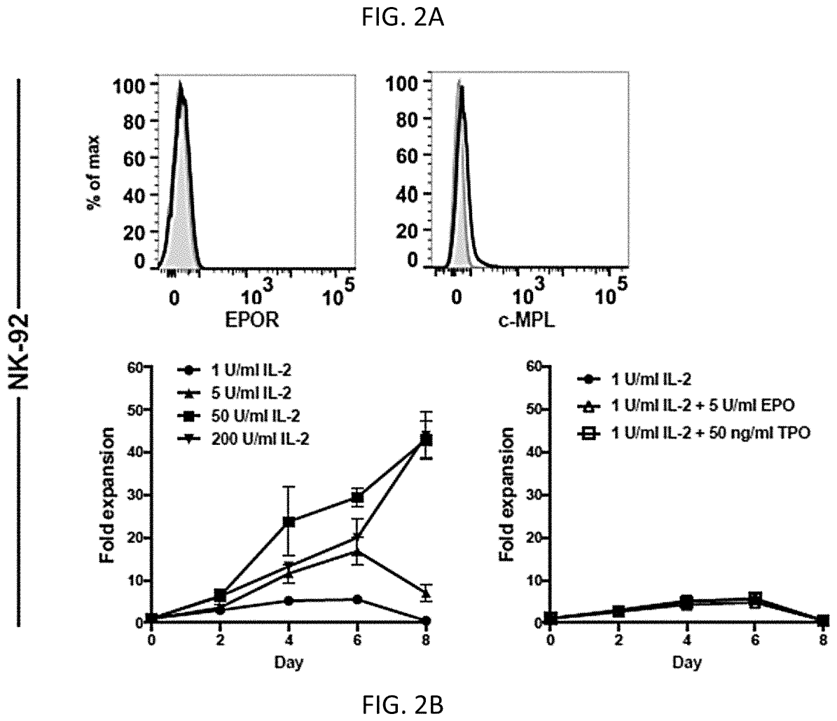

To investigate the functional effects of exogenously expressing EPOR or c-MPL in NK cells, the IL-2-dependent human NK cell line NK-9233-36 was transduced using lentiviral vectors to stably express EPOR or c-MPL along with an EGFP marker (FIGS. 1A-1E). Untransduced parental NK-92 lacked EPOR and c-MPL expression by flow cytometry staining (FIG. 2A). Following transduction, NK cell expansion in response to different doses of IL-2 was examined. Both EPOR⁺ NK-92 and c-MPL⁺ NK-92 cells proliferated in a dose dependent fashion to varying concentrations of IL-2 (FIGS. 1B and 1F).

Introduction of soluble EPO to cultures of EPOR⁺ NK-92 supported the growth of NK cells independent of IL-2, with a 20-fold expansion occurring by day 8 (FIG. 1C). In addition, the combination of EPO and low dose (LD) IL-2 (1 U/ml or 5 U/ml) enhanced the proliferative response of EPOR⁺ NK-92 NK cells compared with LD IL-2 alone. For example, on day 8, the addition of EPO plus IL-2 resulted in 60-times greater proliferation of EPOR⁺ NK-92 cells compared to that observed with 1 U/ml of IL-2 alone (24.5- versus 0.41-fold; p<0.0001) (FIG. 1C). This effect was also evident when the IL-2 dose was increased, where the addition of EPO plus 5 U/ml of IL-2 resulted in a 9-times greater proliferation compared to 5 U/ml of IL-2 alone (43.75-versus 4.75-fold; p<0.0001) (FIG. 1D).

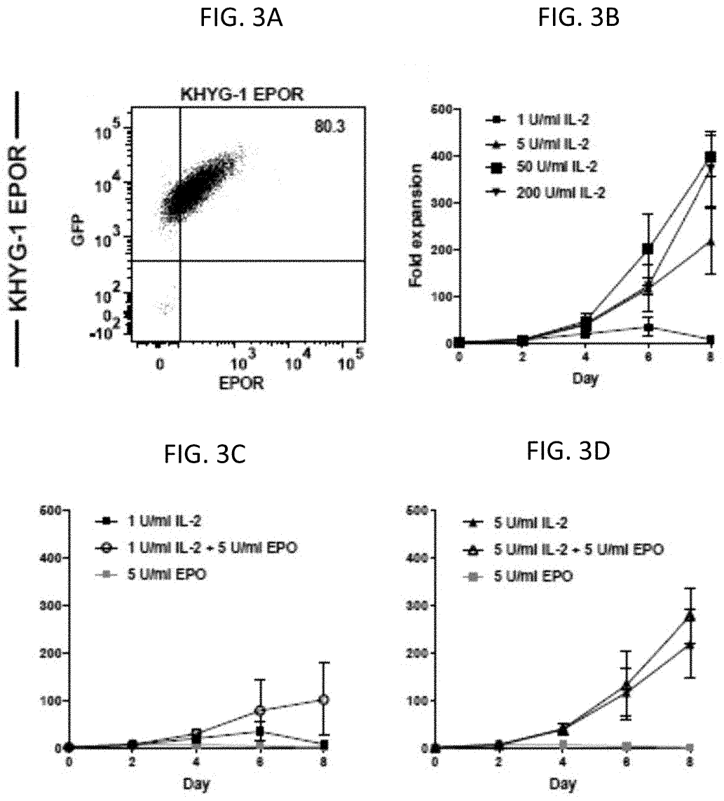

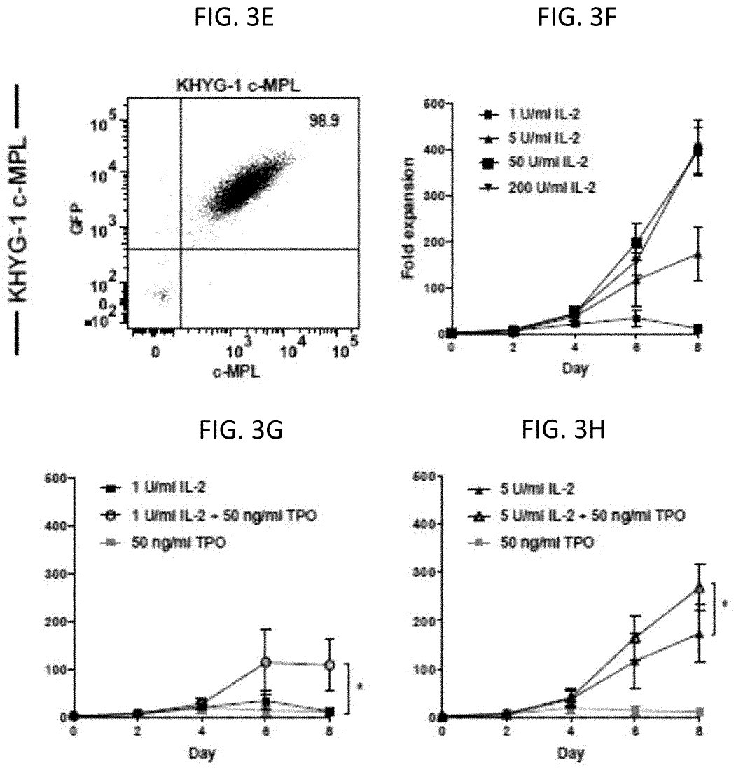

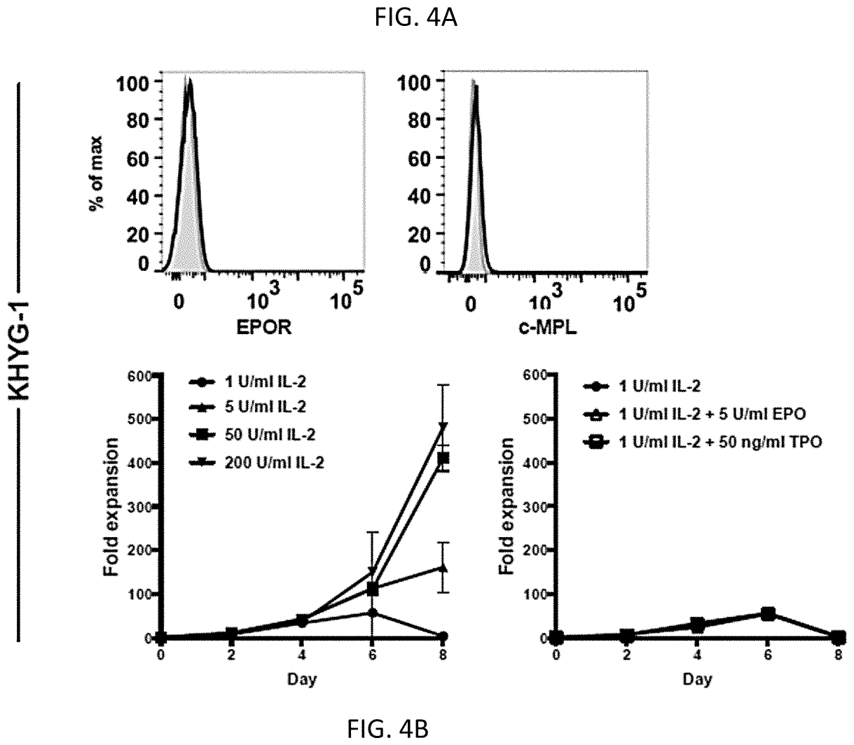

In a similar fashion, c-MPL⁺ NK-92 showed IL-2-independent proliferation in cultures supplemented with TPO alone (7-fold expansion on day 8) (FIG. 1G). Furthermore, adding TPO to LD IL-2 significantly enhanced c-MPL⁺ NK-92 proliferation compared to IL-2 alone or TPO alone (FIGS. 1G and 1H). For example, the addition of TPO plus 1 U/ml IL-2 increased the c-MPL⁺ NK-92 expansion to 23.25-fold compared to only 0.69-fold with 1 U/ml of IL-2 alone (day 8; p=0.01) (FIG. 1G). EPO and TPO administration did not alter the expansion of the parental NK-92 cell line (FIG. 2B). Analogous results were obtained using the KHYG-1 NK cell line transfected to express EPOR or c-MPL receptors (FIGS. 3-4), although in these experiments, EPO alone or TPO alone were insufficient to maintain cell growth of transduced cells (FIG. 3). However, EPO or TPO administration synergized with low-dose IL-2 (1 U/ml or 5 U/ml) to enhance the proliferative response of receptor-transduced KHYG-1 NK cells (FIG. 3). Taken altogether, these data show that exogenously expressed EPOR and c-MPL receptors have the capacity to augment the proliferation of NK cell lines in response to stimulation with their cognate ligands.

### Example 3

### EPOR and c-MPL Expressing NK-92 Cells Up-Regulate the Anti-Apoptotic Proteins Bcl-2 and Bcl-xL Upon EPO or TPO Stimulation Enhancing their Survival

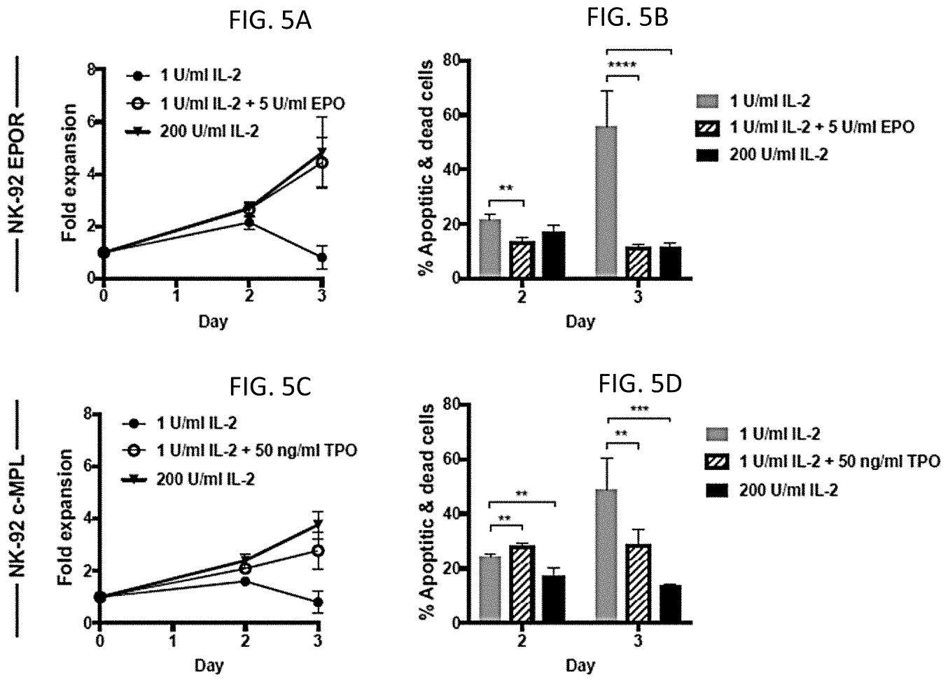

EPOR⁺ and c-MPL⁺ NK-92 cells were used for subsequent studies to characterize in more detail the consequences of ligating these exogenous receptors. Simultaneous short-term cell expansion and apoptosis assays examined the mechanisms underlying EPOR and c-MPL-induced increases in NK cell numbers (FIGS. 5A and 5C). By day 3 of culture, NK cells maintained in LD IL-2 alone showed an increasing number of dead or apoptotic cells, determined by flow cytometry (FIGS. 5B and 5D). In contrast, cultures containing a combination of either EPO or TPO plus LD IL-2 or higher doses of IL-2 (200 U/ml) alone showed significantly lower proportions of dead/apoptotic cells (FIGS. 5B and 5D). These results mirrored the total number of cells present in these cultures (FIGS. 5A and 5C).

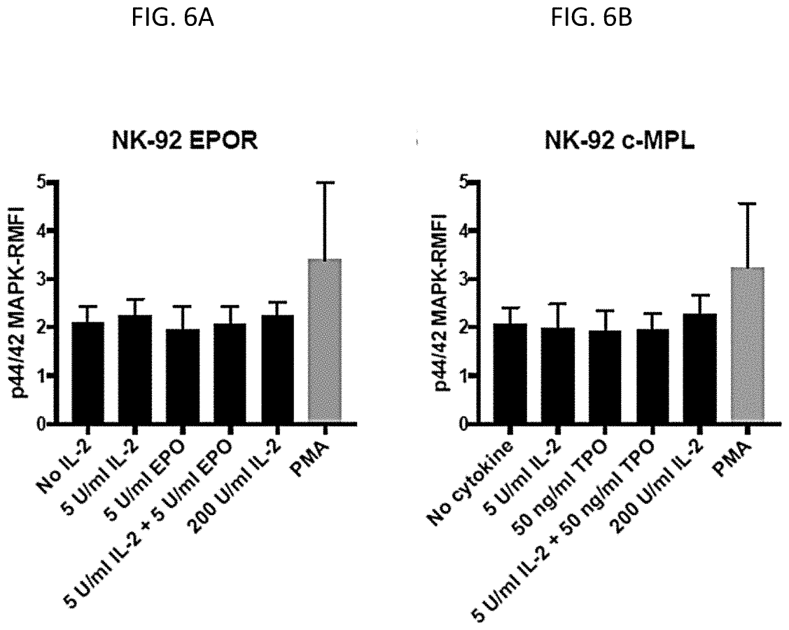

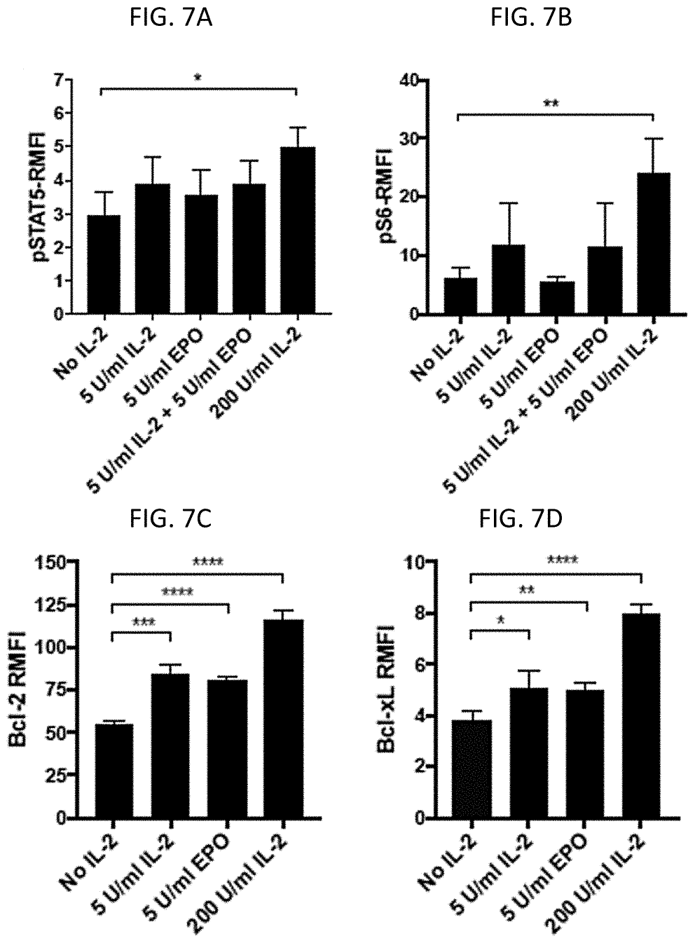

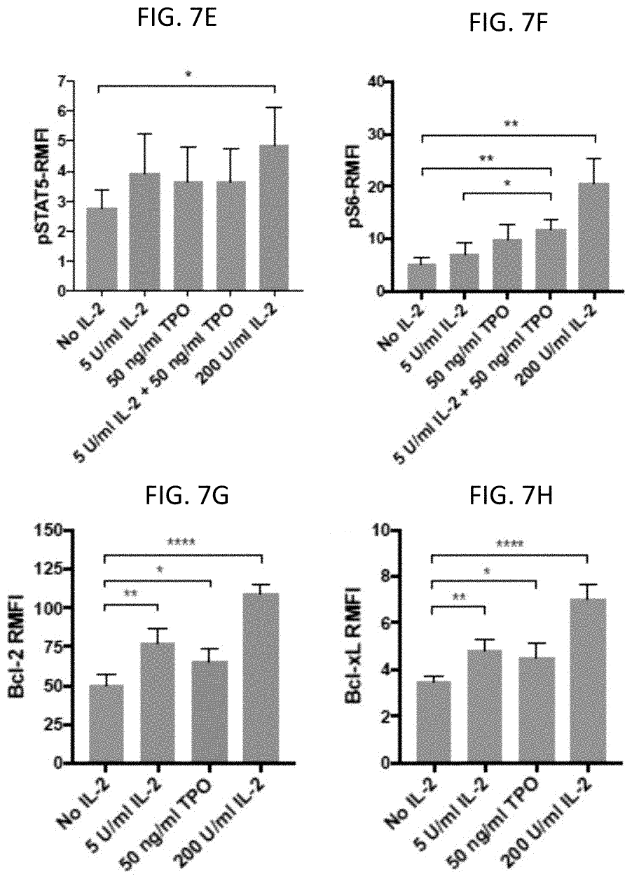

Anti-apoptotic proteins, especially Bcl-xL, have been shown to be regulated by EPO/EPOR and TPO/c-MPL signaling in erythroid progenitors and megakaryocytic cells, respectively. Therefore, transduced NK-92 cells were queried for down-stream signaling pathways including Bcl-2 and Bcl-xL. EPOR and c-MPL have been shown to signal via three main pathways: JAK-STAT, PI3K-AKT and MAPK, which can be assessed using antibodies recognizing pSTAT5, pS6 and phospho-p44/42 MAPK. No alteration of phospho-p44/42 MAPK expression was detected in EPOR and c-MPL⁺ NK-92 cells when cultures were supplemented with IL-2, EPO or TPO (FIGS. 6A and 6B). However, the addition of IL-2 increased pSTAT5 and pS6 levels in a dose dependent manner in both EPOR and c-MPL⁺ NK-92 cells, reaching statistical significance when comparing IL-2 at the 200 U/ml concentration compared with no IL-2 cohorts (FIGS. 7A, 7B, 7E, and 7F). In comparison with the no IL-2 control group, the addition of EPO or TPO appeared to increase pSTAT5 expression on EPOR⁺ NK-92 or c-MPL⁺ NK-92 cells in a fashion comparable to LD IL-2 (FIGS. 7A and 7E). No additive effects on STAT5 phosphorylation were detected when EPO or TPO were combined with LD IL-2 in both EPOR and c-MPL⁺ NK-92 cells (FIGS. 7A and 7E). There was a trend towards increased expression of pS6 in TPO stimulated c-MPL⁺ NK-92 compared to the LD IL-2 group (FIG. 7F). Moreover, adding TPO to LD IL-2 significantly increased pS6 expression compared with LD IL-2 alone (11.84 vs 7.16, p=0.04) (FIG. 7F). These effects on pS6 were not observed in EPO treated EPOR+ NK-92 cells (FIG. 7B), possibly reflecting a difference in signaling potential between the two receptors.

The Bcl-2 and Bcl-xL proteins were investigated as potential target proteins of EPOR and c-MPL signaling in NK cells. As shown in FIGS. 7C, 7D, 7G, and 7H, both low and higher doses of IL-2, EPO and TPO all significantly up-regulated these anti-apoptotic proteins on EPOR⁺ and c-MPL⁺ NK-92 cells. EPO administration stimulated EPOR+ NK-92 cells to produce significantly more Bcl-2 and Bcl-xL proteins than no IL-2 control group (Bcl-2 RMFI: 80.67 vs 54.46, p<0.0001 and Bcl-xL RMFI: 4.92 vs 3.75, p=0.007, respectively) (FIGS. 7C and 7D). Likewise, the addition of TPO activated c-MPL⁺ NK-92 cells to express significantly higher levels of Bcl-2 and Bcl-xL proteins (Bcl-2 RMFI: 64.54 vs 50.43, p=0.03 and Bcl-xL RMFI: 4.52 vs 3.46, p=0.016) compared to the no IL-2 control group (FIGS. 7G and 7H).

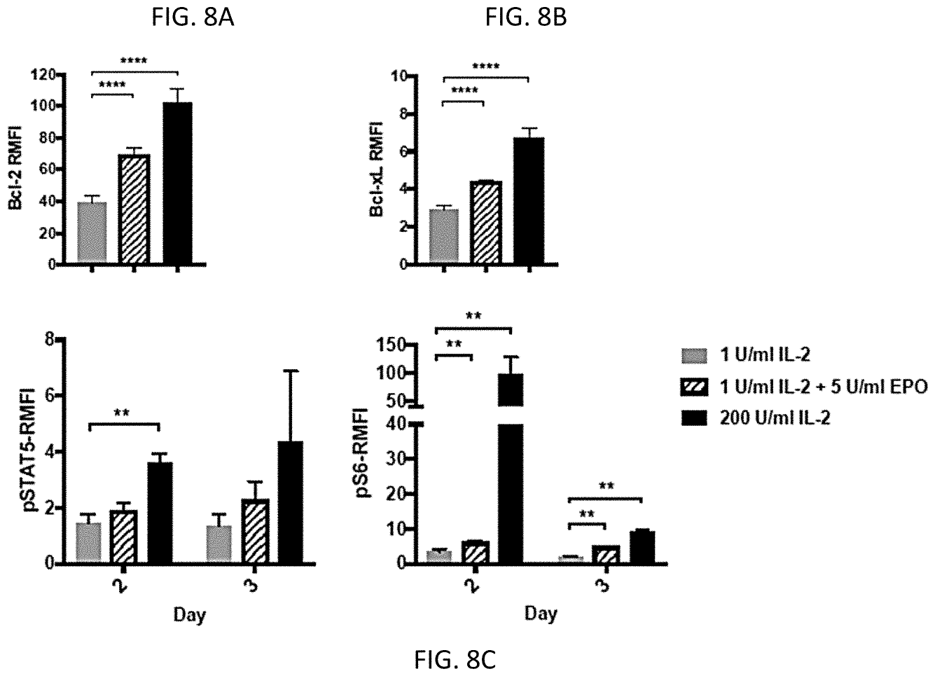

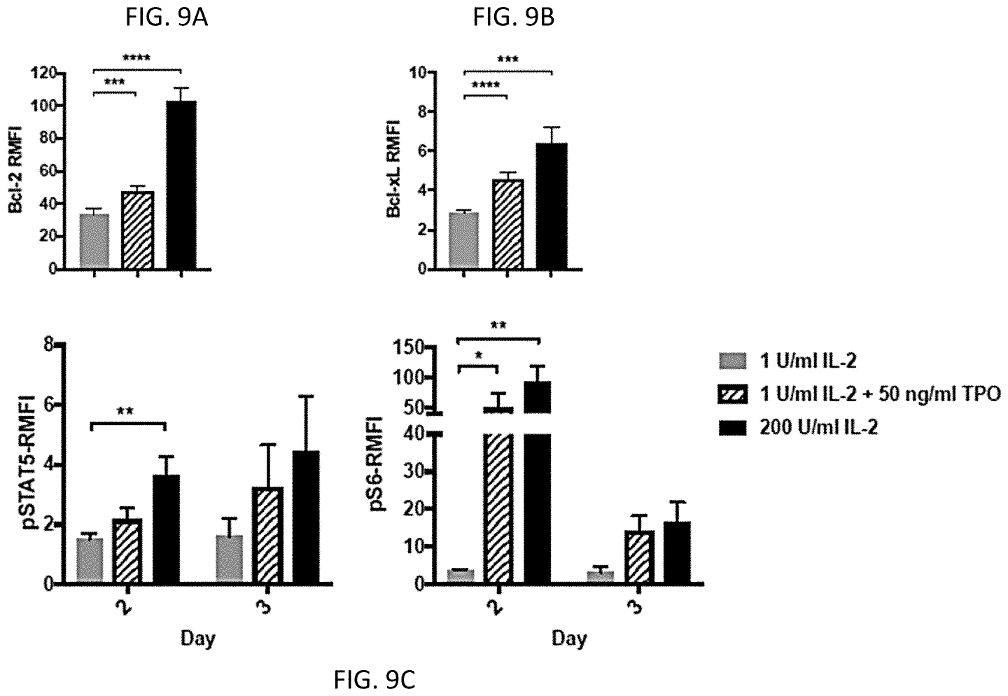

These results suggest that hematopoietic growth factor receptors exogenously expressed in NK-92 cells can signal to up-regulate the anti-apoptotic proteins Bcl-2 and Bcl-xL, which together prolong cell survival. In EPOR+ NK-92 cells, this signaling may proceed through STAT5, while MPL⁺ NK-92 cells may utilize both STAT5 and AKT-S6 pathways. Similar results were obtained in experiments querying other time points of the cell culture (FIGS. 8A-8C and 9A-9C).

### Example 4

### Expression of c-MPL in NK-92 Cells Augments their Killing of Raji Tumor Cells in the Presence of TPO

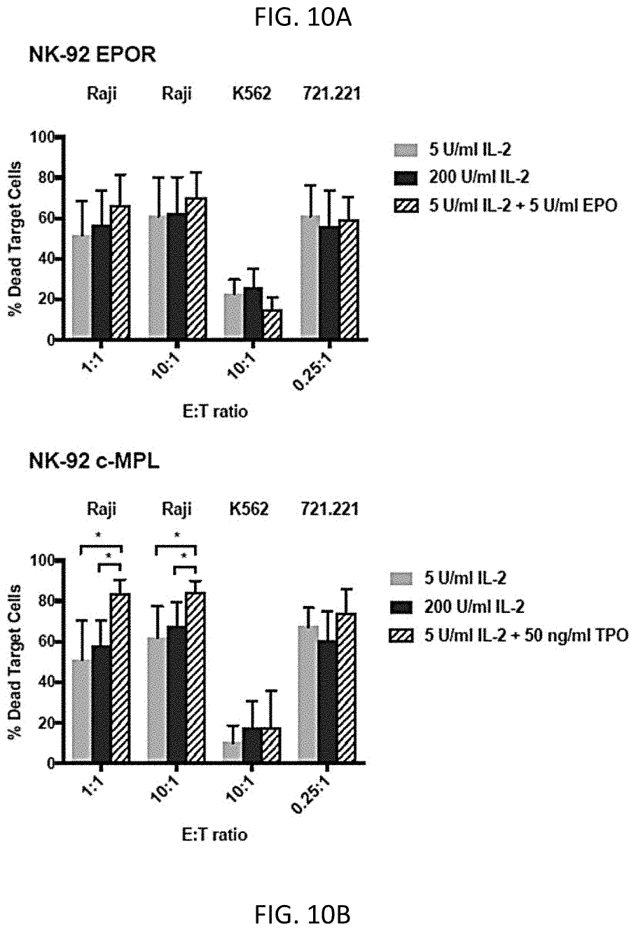

In order to test the functional consequences of expressing EPO or TPO receptors, transgenic EPOR⁺ and c-MPL⁺ NK-92 cells were expanded in the presence of EPO or TPO and cytotoxicity assays were performed with three different tumor cell lines (Raji, K562, and 721.221). Compared to parental controls, no deficit in the killing capacity of transduced NK-92 cells expanded in presence of EPO or TPO was observed (data not shown). Compared to LD IL-2 alone, there was a trend towards higher Raji killing by EPOR⁺ NK-92 cells stimulated with EPO and LD IL-2, while killing between LD IL-2 and EPO stimulated EPOR⁺ NK-92 cells against 721.221 or K562 tumor cell lines was similar (FIG. 10A). However, there was a striking increase in Raji tumor killing by c-MPL⁺ NK-92 cells exposed to TPO plus LD IL-2 compared to IL-2 alone at both lower and higher doses; the mean percentages of dead Raji cells in TPO+LD IL-2 cohorts were 84% (1:1) and 85% (10:1) compared with LD IL-2 alone (51% (1:1), p=0.02 and 62% (10:1), p=0.03) and the higher dose of IL-2 (200 U/ml) alone (58% (1:1), p=0.01 and 67% (10:1), p=0.04), respectively (FIG. 10B). In contrast, no significant killing augmentation against 721.221 or K562 tumor targets was observed with TPO administration (FIG. 10B).

### Example 5

### Transgenic Expression of c-MPL in Primary NK Cells Promotes Proliferation in Presence of TPO

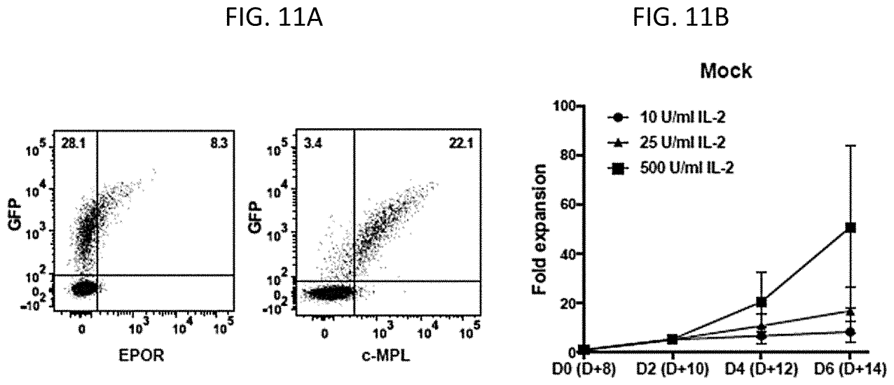

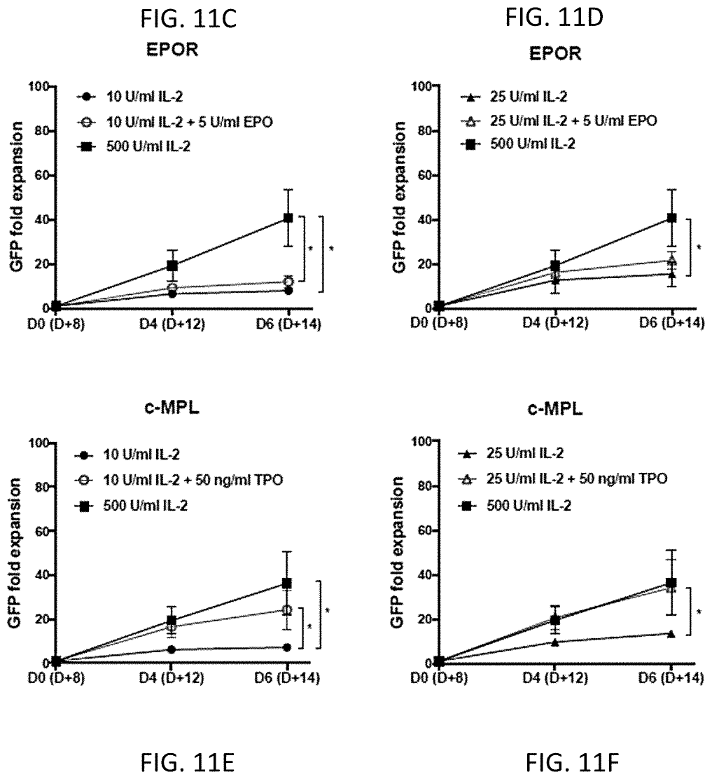

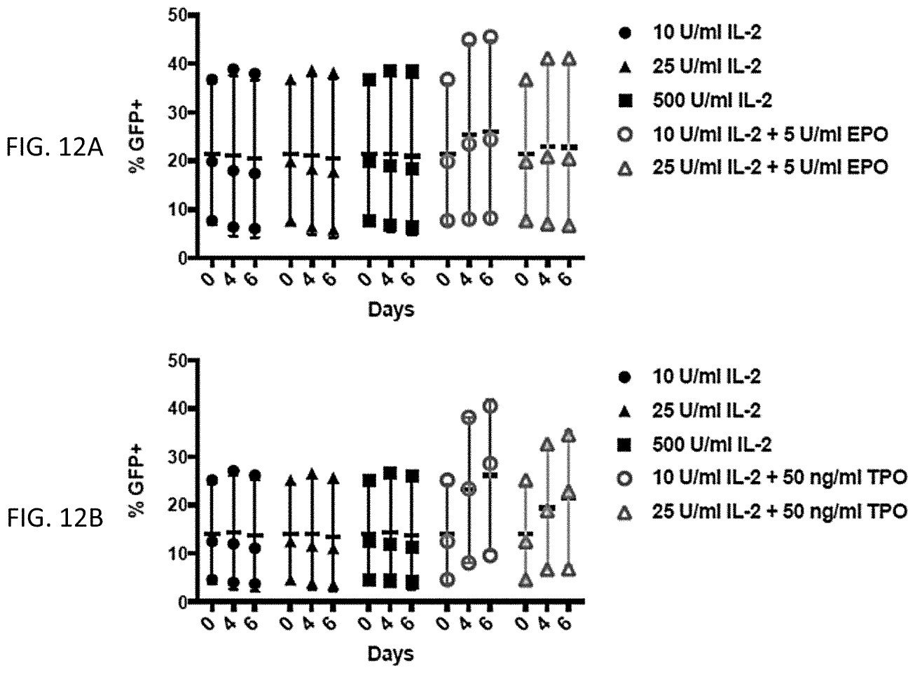

Data from the above experiments indicated proliferative, survival, and functional benefits of over-expressing EPOR or c-MPL in NK 92 cells in the presence of EPO or TPO and LD IL-2. Therefore, using the same lentiviral constructs as used to transduce NK cell lines, 4-37% of primary NK cells (activated before transduction with IL-2 plus an EBV-transformed lymphoblastic cell line) were successfully transduced to express either EPOR/GFP or c-MPL/GFP (FIG. 11A, FIG. 12). Similarly, activated but untransduced (Mock) NK cells were used as controls and expanded in an IL-2 concentration-dependent fashion (FIG. 11B). During a 6 day competitive expansion culture, c-MPL-transduced NK cells supplemented with TPO plus LD IL-2 showed an increase in the relative percentage of the GFP⁺ NK cell population, which is consistent with the c-MPL⁺ NK cells having a TPO-induced proliferative advantage (FIG. 12B). This was further quantified by characterizing the fold expansion of GFP⁺ (transduced) cells. In the presence of 10 U/ml LD IL-2, the addition of TPO increased the fold change of GFP⁺ c-MPL⁺ NK cells from 7.2-fold to 24.3-fold (p=0.03) (FIG. 11E). With a slightly higher concentration of IL-2 (25 U/ml), the impact of TPO was further magnified, increasing the GFP⁺ fold change from 13.5-fold to 34.1-fold (p=0.047) (FIG. 11F). The proliferation level with TPO+25 U/ml IL-2 (34.1-fold) was comparable to that observed with much higher doses of IL-2 (500 U/ml) (36.3-fold) (p=0.85). In contrast, EPOR⁺ NK cells did not show an increase in the mean GFP⁺ fold-expansion in the presence of EPO plus LD IL-2 compared with LD IL-2 alone (FIGS. 11C and 11D), perhaps because there was a lower level of EPOR expression on the surface of transduced primary NK cells (FIG. 11A) or different EPOR signaling capacity in primary NK cells compared with the NK-92 cell line. Nonetheless, c-MPL expression in primary NK cells clearly enhanced their ability to proliferate.

### Example 6

### c-MPL-Transduced Primary NK Cells Show Increased Anti-Tumor Function Upon TPO Stimulation

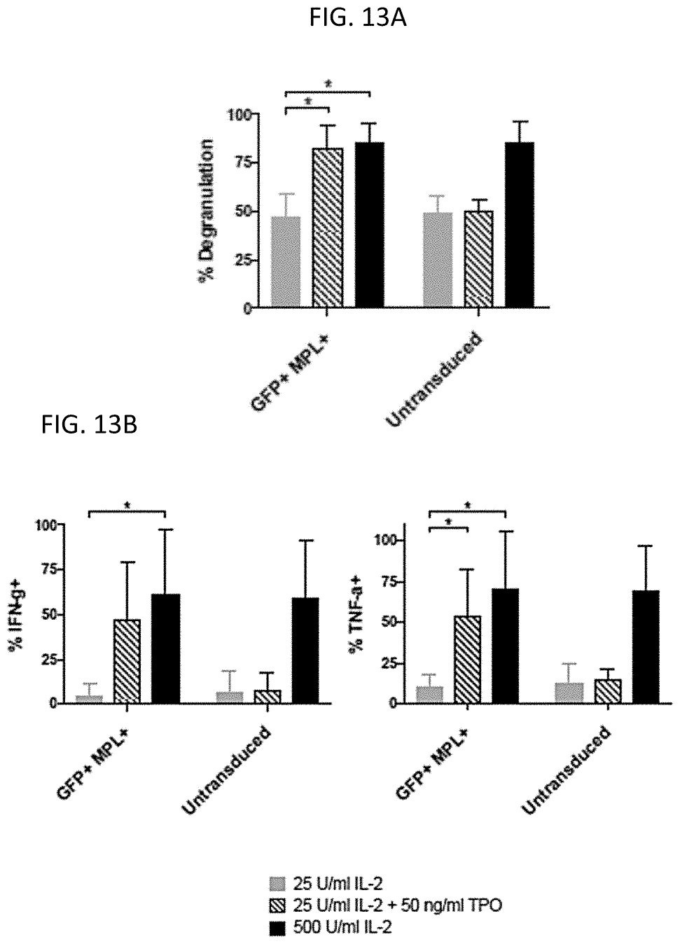

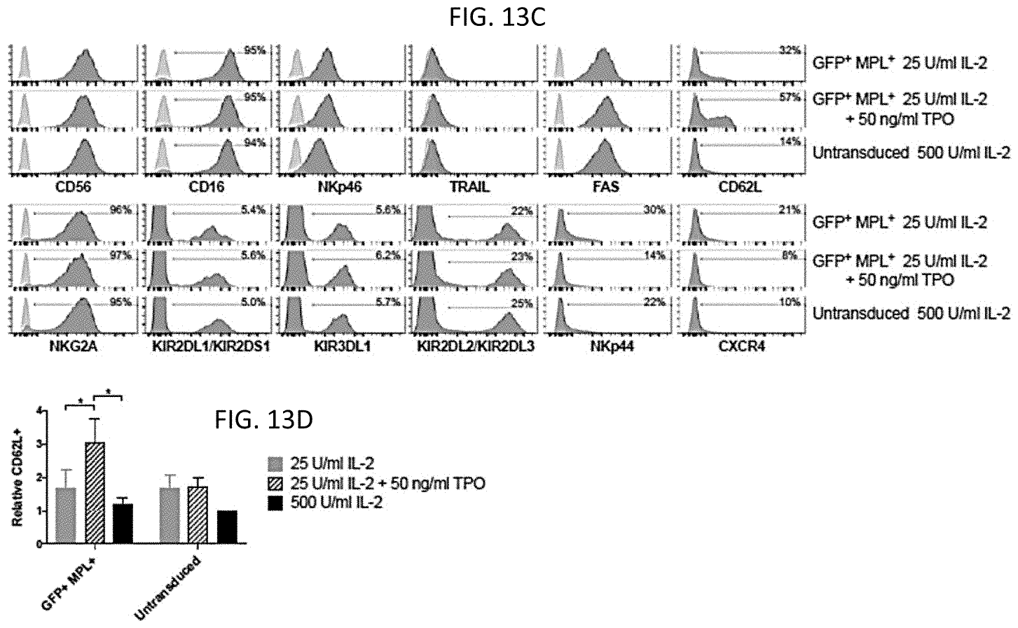

To investigate the effects on NK cell function, primary NK cells were transduced with a c-MPL/GFP encoding lentiviral vector, cultured for four days in different cytokine combinations, then were co-cultured with K562 target cells. CD107a degranulation and cytokine production were investigated in GFP⁺ transduced cells and untransduced controls. There was a significantly higher amount of degranulation in GFP⁺ MPL⁺ cells cultured in media with TPO and 25 U/ml IL-2 (FIG. 13A) compared to 25 U/ml of IL-2 alone. Strikingly, the level of cellular degranulation observed in these cells with TPO and 25 U/ml IL-2 was comparable to that observed with a much higher dose of IL-2 (500 U/ml); degranulation in GFP⁺ MPL⁺ NK cells averaged 48% with 25 U/ml of IL-2 alone, 83% with the addition of TPO, and 86% with the higher 500 U/ml IL-2 dose (FIG. 13A). Similarly, the percent of GFP⁺ MPL⁺ transduced NK cells that produced IFN-γ or TNF-α when co-cultured with K562 cells was considerably augmented by the addition of TPO to 25 U/ml IL-2 compared with 25 U/ml IL-2 alone (FIG. 13B). For this comparison, p=0.04 for IFN-γ production, p=0.01 for TNF-α production, using data normalized to cytokine production of untransduced control cells in 500 U/ml IL-2 to mitigate inter-donor variation (not shown). Administration of TPO had no measured effects on degranulation or cytokine production of untransduced control cells (FIGS. 13A and 13B). These results clearly indicate that c-MPL receptors that are exogenously expressed in primary human NK cells can augment commonly measured anti-tumor functions in response to ligand stimulation.

The overall NK cell phenotype of the c-MPL-transduced primary NK cells was also investigated using a number of antibodies recognizing NK cell receptors and molecules important for NK cell function. With the exception of CD62L (L-selectin), no phenotypic differences were observed in gated GFP⁺ (MPL expressing) NK cells when TPO was added to media containing 25 U/ml of IL-2 (FIG. 13C). The overall percentage of NK cells expressing the lymphoid-tissue homing receptor CD62L (L-selectin) was higher in GFP⁺ MPL⁺ transduced cells when TPO was added to media containing 25 U/ml of IL-2 compared with IL-2 alone at either at 25 or 500 U/ml concentration (FIGS. 13C and 13D). Although the overall percentage of CD62L positivity varied considerably between donors, when normalized to each donor, supplementing media with TPO significantly increased CD62L expression in GFP⁺ MPL⁺ transduced cells but not untransduced cells (FIG. 13D).

### Example 7

### Primary Human NK Cells Genetically Modified to Express c-MPL Preferentially Persist Upon Adoptive Transfer into Immunodeficient Mice Receiving TPO

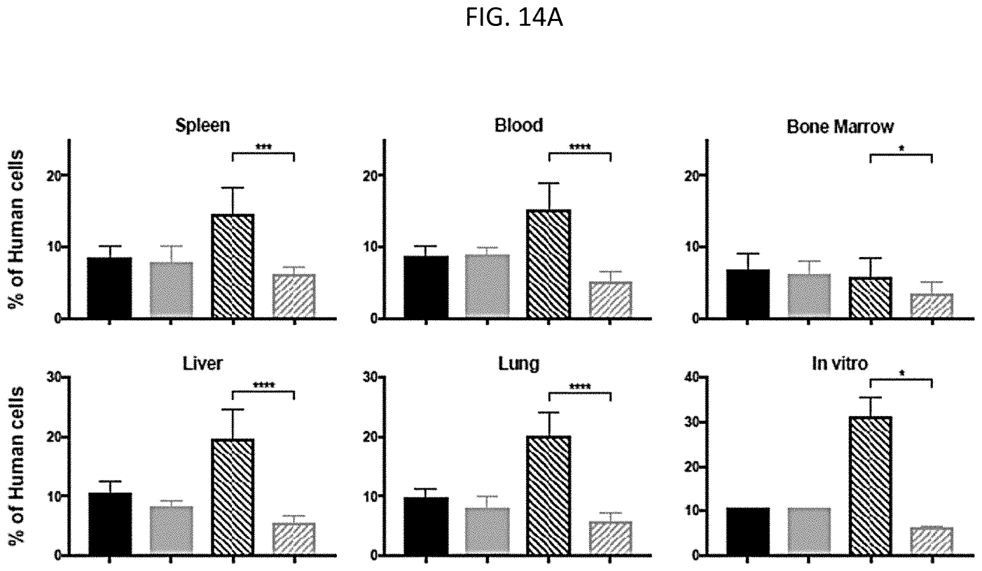

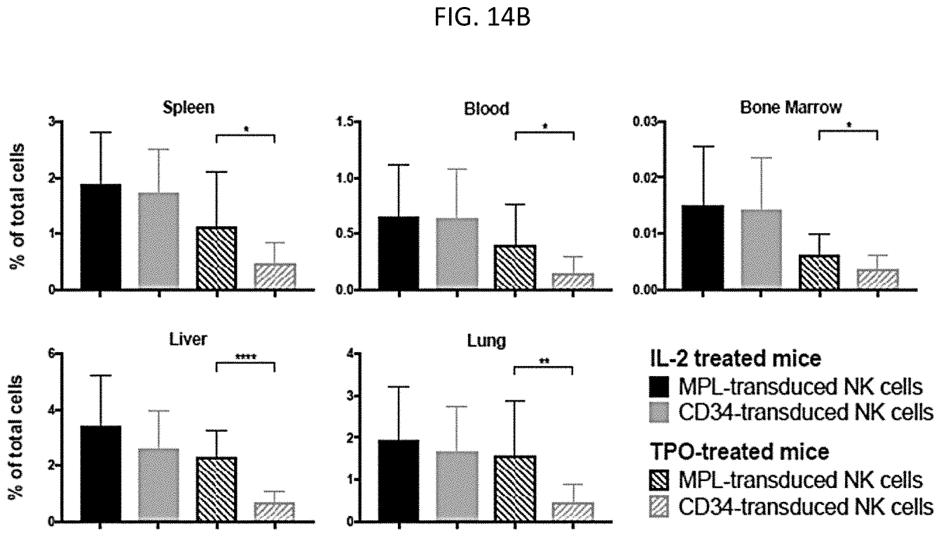

The capacity for exogenous c-MPL receptor expression to support adoptively transferred NK cells in vivo was examined. Primary human NK cells transduced with lentiviral constructs encoding GFP and either c-MPL or truncated surface expressed human CD34 (as a control) were mixed at a 1:1 ratio, then were injected intravenously into NOD SCID IL2rg^{-/-} (NSG) immunodeficient mice. Following NK cell infusion, mice received daily intraperitoneal injections of TPO (50 μg/kg) or human IL-2 (10,000 IU/mouse; a dose previously reported to represent “low dose IL-2” treatment in vivo) (Granzin et al., Oncoimmunology 5:e1219007, 2016). After 4 days, GFP⁺ MPL⁺ cells represented a significantly higher proportion of human cells detected in all the organs of mice that had received TPO (FIG. 14A). In contrast, mice which had been administered IL-2 showed roughly equal proportions of GFP⁺ MPL⁺ and GFP CD34⁺ cells (FIG. 14A). GFP⁺ MPL⁺ cells constituted approximately 10% of the mixed cells that were administered to mice. However, in mice that received TPO, the average percentage of GFP⁺ MPL⁺ cells rose amongst the recovered human cells to 15% in blood and spleen, and 20% in the liver and lung. These data suggest that MPL-expressing NK cells achieved a selective persistence advantage in vivo when TPO was administered, in comparison with both control CD34-transduced cells and bystander untransduced NK cells. Parallel cultures of the same mixed cells supplemented in vitro with only TPO showed an analogous increase in the GFP⁺ MPL⁺ population to 31% (FIG. 14A). For a less comparative measure of the numbers of in vivo persisting NK cells, populations were quantitated as a percentage of the total live cells (mouse or human) isolated from each organ (FIG. 14B). This analysis similarly revealed that TPO-treated mice had significantly more GFP⁺ MPL cells compared to GFP⁺ CD34⁺ control cells (FIG. 14B); in TPO-treated mice, GFP⁺ CD34⁺ cells would not be expected to receive any exogenous cytokine support. Importantly, GFP⁺ MPL cells in TPO-treated mice approached percentages observed in IL-2 treated mice (FIG. 14B). These data suggest that MPL ligation on transduced NK cells preferentially supported their persistence in vivo in a fashion comparable to IL-2, although perhaps somewhat less effective under these experimental conditions.

### Example 8

### Primary T Cells Transduced to Express EPOR and c-MPL have Augmented Cell Numbers in the Presence of Ligand

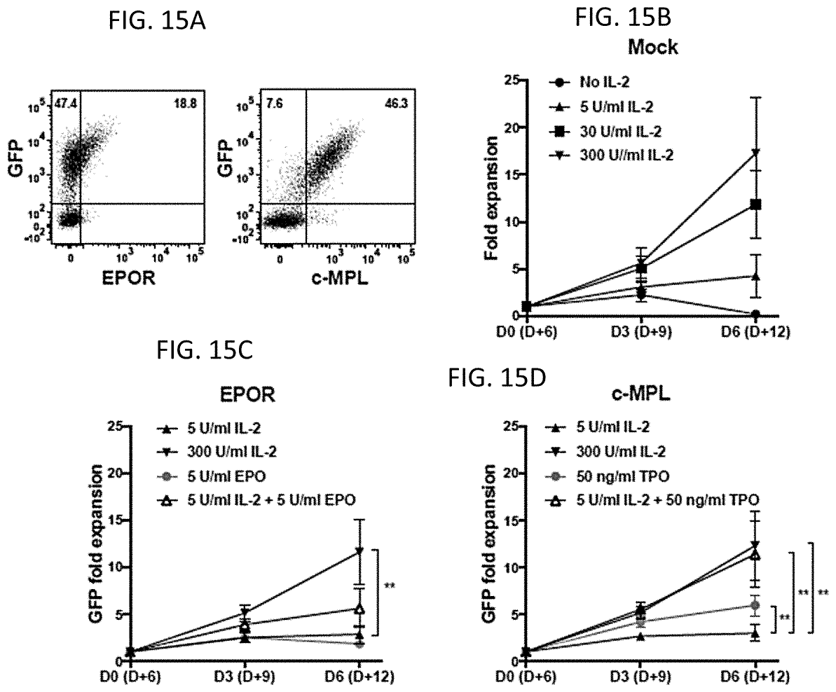

The effects of transgenic expression of EPOR and c-MPL were also evaluated in primary human T cells. Efficient transduction of EPOR/GFP or c-MPL/GFP in activated T cells from healthy donors was observed, ranging from 40 to 60% positive (FIG. 15A). Activated but untransduced (Mock) T cells were used as controls and were observed to proliferate in vitro in an IL-2 concentration-dependent fashion (FIG. 15B). A 6 day competitive culture was used to examine the fold expansion of GFP⁺ (transduced) cells. EPOR T cells in the presence of EPO plus LD IL-2 appeared to show a slightly higher mean GFP⁺ fold-expansion compared with LD IL-2 alone (D6; 5.61 vs 2.81-fold, p=0.05) (FIG. 15C). c-MPL transduced T cells in the presence of TPO alone appeared to show acquisition of an IL-2 independent expansion capacity; the GFP⁺ fold-expansion on D6 was 5.96-fold in the presence of TPO alone versus 2.99-fold in the presence of LD IL-2 (5 U/ml) alone (p=0.006). In addition, the relative percentage of c-MPL⁺ activated T cells increased on day 6 to 11.39-fold when exposed to TPO plus LD IL-2 (p=0.004 compared to LD IL-2 alone) (FIG. 15D). Remarkably, the level of T-cell proliferation with TPO+LD IL-2 was comparable to that achieved with much higher doses of IL-2 (300 U/ml). Taken altogether, transgenic expression of both EPOR and c-MPL appeared to have functional activity when expressed in primary T cells, but this effect was quantitatively larger with c-MPL.

### Example 9

### c-MPL-Expressing Human NK Cells Proliferate in Response to Drug Mimetics of Thrombopoietin

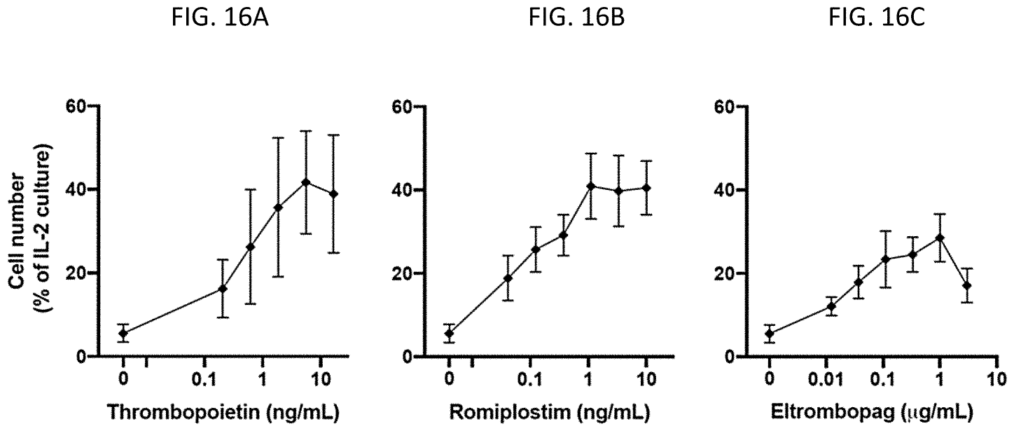

NK cells from PBMC from three blood donors were stimulated and transduced to express GFP and c-MPL, then were cultured for 4-5 days with thrombopoietin, romiplostim, or eltrombopag. Numbers of GFP cells were enumerated and expressed as a percentage of numbers obtained from parallel cultures containing 500 U/mL IL-2 (FIGS. 16A-16C). These data show that besides thrombopoietin, romiplostim and eltrombopag can also be used to induce proliferation of c-MPL transduced NK cells.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A modified natural killer (NK) cell or T cell expressing a heterologous erythropoietin receptor (EPOR) protein.

2. The modified NK cell or T cell of claim 1, wherein the modified NK cell or T cell further expresses a chimeric antigen receptor.

3. The modified NK cell or T cell of claim 1, wherein the EPOR protein is a human EPOR protein.

4. The modified NK cell or T cell of claim 1, wherein the human EPOR protein comprises at least 95% sequence identity to the amino acid sequence of SEQ ID NO: 3.

5. The modified NK cell or T cell of claim 4, wherein the human EPOR protein comprises the amino acid sequence of SEQ ID NO: 3.

6. The modified NK cell or T cell of claim 1, wherein the cell comprises a nucleic acid molecule encoding the EPOR protein.

7. The modified NK cell or T cell of claim 1, wherein the NK cell or T cell is transduced or transfected with a nucleic acid encoding the EPOR protein.

8. The modified NK cell or T cell of claim 1, wherein the NK cell is a human NK cell or the T cell is a human T cell.

9. A method of expanding modified NK cells or T cells, comprising culturing the modified NK cell or T cell of claim 1 in the presence of an erythropoietin receptor agonist.

10. The method of claim 9, further comprising culturing the modified NK cells or T cells in the presence of interleukin (IL)-2.

11. The method of claim 10, wherein the modified NK cell or T cell is cultured in the presence of 1-50 U/ml IL-2.

12. A method of treating a subject with cancer, comprising:
administering the modified NK cell or T cell of claim 1 to the subject; and
administering an EPOR agonist to the subject.

13. The method of claim 12, wherein the EPOR agonist is erythropoietin or darbepoetin.

14. The method of claim 12, further comprising administering IL-2 to the subject.

15. The method of claim 14, wherein the IL-2 is administered at about 0.5-4 million units/m².

16. The method of claim 12, wherein the modified NK cell or T cell is contacted with IL-2 prior to administering to the subject.

17. The method of claim 16, wherein the modified NK cell or T cell is contacted with 1-50 U/ml IL-2.

18. The method of claim 12, wherein the subject has lymphoma, leukemia, renal cell cancer, breast cancer, colon cancer, bladder cancer, or a uroepithelia carcinoma.


SEQUENCE LISTING (6 sequences)

SEQ ID NO: 1 - protein, 635 aa, Homo sapiens
MPSWALFMVTSCLLLAPQNLAQVSSQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 2 - DNA, 1908 bp, Homo sapiens
  source 1..1908: mol_type = unassigned DNA
atgccctcctgggccctcttcatggtcacctcctgcctcctcctggcccctcaaaacctggcccaagtcagcagccaagatgtctccttgctggcatcagactcagagcccctgaagtgtttctcccgaacatttgaggacctcacttgcttctgggatgaggaagaggcagcgcccagtgggacataccagctgctgtatgcctacccgcgggagaagccccgtgcttgccccctgagttcccagagcatgccccactttggaacccgatacgtgtgccagtttccagaccaggaggaagtgcgtctcttctttccgctgcacctctgggtgaagaatgtgttcctaaaccagactcggactcagcgagtcctctttgtggacagtgtaggcctgccggctccccccagtatcatcaaggccatgggtgggagccagccaggggaacttcagatcagctgggaggagccagctccagaaatcagtgatttcctgaggtacgaactccgctatggccccagagatcccaagaactccactggtcccacggtcatacagctgattgccacagaaacctgctgccctgctctgcagaggcctcactcagcctctgctctggaccagtctccatgtgctcagcccacaatgccctggcaagatggaccaaagcagacctccccaagtagagaagcttcagctctgacagcagagggtggaagctgcctcatctcaggactccagcctggcaactcctactggctgcagctgcgcagcgaacctgatgggatctccctcggtggctcctggggatcctggtccctccctgtgactgtggacctgcctggagatgcagtggcacttggactgcaatgctttaccttggacctgaagaatgttacctgtcaatggcagcaacaggaccatgctagctcccaaggcttcttctaccacagcagggcacggtgctgccccagagacaggtaccccatctgggagaactgcgaagaggaagagaaaacaaatccaggactacagaccccacagttctctcgctgccacttcaagtcacgaaatgacagcattattcacatccttgtggaggtgaccacagccccgggtactgttcacagctacctgggctcccctttctggatccaccaggctgtgcgcctccccaccccaaacttgcactggagggagatctccagtgggcatctggaattggagtggcagcacccatcgtcctgggcagcccaagagacctgttatcaactccgatacacaggagaaggccatcaggactggaaggtgctggagccgcctctcggggcccgaggagggaccctggagctgcgcccgcgatctcgctaccgtttacagctgcgcgccaggctcaacggccccacctaccaaggtccctggagctcgtggtcggacccaactagggtggagaccgccaccgagaccgcctggatctccttggtgaccgctctgcatctagtgctgggcctcagcgccgtcctgggcctgctgctgctgaggtggcagtttcctgcacactacaggagactgaggcatgccctgtggccctcacttccagacctgcaccgggtcctaggccagtaccttagggacactgcagccctgagcccgcccaaggccacagtctcagatacctgtgaagaagtggaacccagcctccttgaaatcctccccaagtcctcagagaggactcctttgcccctgtgttcctcccaggcccagatggactaccgaagattgcagccttcttgcctggggaccatgcccctgtctgtgtgcccacccatggctgagtcagggtcctgctgtaccacccacattgccaaccattcctacctaccactaagctattggcagcagccttga

SEQ ID NO: 3 - protein, 508 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 4 - DNA, 1527 bp, Homo sapiens
  source 1..1527: mol_type = unassigned DNA
atggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatggacactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttag

SEQ ID NO: 5 - DNA, 9109 bp, synthetic construct
  misc_feature 1..9109: note = Synthetic vector including TPO receptor
  source 1..9109: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagccacgaacttctctctgttaaagcaagcaggagatgttgaagaaaaccccgggcctatgccctcctgggccctcttcatggtcacctcctgcctcctcctggcccctcaaaacctggcccaagtcagcagccaagatgtctccttgctggcatcagactcagagcccctgaagtgtttctcccgaacatttgaggacctcacttgcttctgggatgaggaagaggcagcgcccagtgggacataccagctgctgtatgcctacccgcgggagaagccccgtgcttgccccctgagttcccagagcatgccccactttggaacccgatacgtgtgccagtttccagaccaggaggaagtgcgtctcttctttccgctgcacctctgggtgaagaatgtgttcctaaaccagactcggactcagcgagtcctctttgtggacagtgtaggcctgccggctccccccagtatcatcaaggccatgggtgggagccagccaggggaacttcagatcagctgggaggagccagctccagaaatcagtgatttcctgaggtacgaactccgctatggccccagagatcccaagaactccactggtcccacggtcatacagctgattgccacagaaacctgctgccctgctctgcagaggcctcactcagcctctgctctggaccagtctccatgtgctcagcccacaatgccctggcaagatggaccaaagcagacctccccaagtagagaagcttcagctctgacagcagagggtggaagctgcctcatctcaggactccagcctggcaactcctactggctgcagctgcgcagcgaacctgatgggatctccctcggtggctcctggggatcctggtccctccctgtgactgtggacctgcctggagatgcagtggcacttggactgcaatgctttaccttggacctgaagaatgttacctgtcaatggcagcaacaggaccatgctagctcccaaggcttcttctaccacagcagggcacggtgctgccccagagacaggtaccccatctgggagaactgcgaagaggaagagaaaacaaatccaggactacagaccccacagttctctcgctgccacttcaagtcacgaaatgacagcattattcacatccttgtggaggtgaccacagccccgggtactgttcacagctacctgggctcccctttctggatccaccaggctgtgcgcctccccaccccaaacttgcactggagggagatctccagtgggcatctggaattggagtggcagcacccatcgtcctgggcagcccaagagacctgttatcaactccgatacacaggagaaggccatcaggactggaaggtgctggagccgcctctcggggcccgaggagggaccctggagctgcgcccgcgatctcgctaccgtttacagctgcgcgccaggctcaacggccccacctaccaaggtccctggagctcgtggtcggacccaactagggtggagaccgccaccgagaccgcctggatctccttggtgaccgctctgcatctagtgctgggcctcagcgccgtcctgggcctgctgctgctgaggtggcagtttcctgcacactacaggagactgaggcatgccctgtggccctcacttccagacctgcaccgggtcctaggccagtaccttagggacactgcagccctgagcccgcccaaggccacagtctcagatacctgtgaagaagtggaacccagcctccttgaaatcctccccaagtcctcagagaggactcctttgcccctgtgttcctcccaggcccagatggactaccgaagattgcagccttcttgcctggggaccatgcccctgtctgtgtgcccacccatggctgagtcagggtcctgctgtaccacccacattgccaaccattcctacctaccactaagctattggcagcagccttgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 6 - DNA, 8728 bp, synthetic construct
  misc_feature 1..8728: note = Synthetic vector including EPO receptor
  source 1..8728: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagccacgaacttctctctgttaaagcaagcaggagatgttgaagaaaaccccgggcctatggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatggacactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt